(19) United States
(12) Patent Application Publication
(10) Pub. No.: US 2020/0132686 A1
(43) Pub. Date: Apr. 30, 2020

(54) ASSAYS FOR ASSESSING NEUTRALIZING ANTIBODIES LEVELS IN SUBJECTS TREATED WITH A BIOLOGICAL DRUG AND USES THEREOF IN PERSONALIZED MEDICINE

(71) Applicant: RAMBAM MED-TECH LTD., Haifa (IL)

(72) Inventors: Yehuda CHOWERS, Tel Aviv (IL); Sigal PRESSMAN, Pardes Hanna (IL); Alexandra BLATT, Haifa (IL); Shiran GERASSY-VAINBERG, Tirat Carmel (IL)

(21) Appl. No.: 16/738,757

(22) Filed: Jan. 9, 2020

Related U.S. Application Data
(63) Continuation-in-part of application No. PCT/IL2018/050753, filed on Jul. 10, 2018.
(60) Provisional application No. 62/530,310, filed on Jul. 10, 2017.

(51) Int. Cl. G01N 33/564; C07K 16/28; G01N 33/543

(52) U.S. Cl. CPC G01N 33/564; C07K 16/2875; G01N 2800/54; G01N 2800/52; G01N 33/543

(57) ABSTRACT

The invention relates to assays, devices and kits for accurate determination of neutralizing antibodies levels in samples of a subject suffering from an immune-mediated disorder, treated with biological drugs, and for predicting responsiveness to the drug in these patients.

### CROSS-REFERENCE TO RELATED APPLICATIONS

The present application is a continuation in part of PCT/IL2018/050753, filed Jul. 10, 2018, which claims the benefit of the filing date of application no. 62/530,310, filed Jul. 10, 2017, both of which are hereby incorporated herein by reference in their entireties.

### FIELD OF INVENTION

The invention relates to personalized medicine. More particularly, the invention provides assays, devices and kits for accurate determination of neutralizing antibodies levels in a subject suffering from an immune-mediated disorder, that is treated with a biological drug.

### BACKGROUND ART

References considered to be relevant as background to the presently disclosed subject matter are listed below:

[1] Baert F, Noman M, Vemeire S, et al. influence of immunogenicity on the long-term efficacy of infliximab in Crohn's disease. N Engl J Med 2003; 348:601-8.

[2] Ordas I, Feagan B G, Sandborn W J. Therapeutic drug monitoring of tumor necrosis factor antagonists in inflammatory bowel disease. Clin. Gastroenterol Hepatol 2012; 10:1079-87; quiz e85-6.

[3] Yanai H, Lichtenstein L, Assa A, et al. Levels of drug and antidrug antibodies are associated with outcome of interventions after loss of response to infliximab or adalimumab. Clin Gastroenterol Hepatol 2015; 13:522-530 e2.

[4] Ungar B, Levy I, Yavne Y, et al. Optimizing Anti-TNF-alpha Therapy: Serum Levels of Infliximab and Adalimumab Are Associated With Mucosal Healing in Patients With Inflammatory Bowel Diseases. Clin Gastroenterol Hepatol 2016; 14:550-557 e2.

[5] Ben-Horin, S. & Chowers, Y. Tailoring anti-TNF therapy in IBD: drug levels and disease activity. Nat. Rev. Gastroenterol, Hepatol. 11, 243-255 (2014).

[6] Weisshof, R. et al. Anti-infliximab Antibodies with Neutralizing Capacity in Patients with Inflammatory Bowel Disease: Distinct Clinical implications Revealed by a Novel Assay. Inflamm. Bowel Dis. (2016).

[7] Kopylov, U. et al. Clinical utility of antihuman lambda chain-based enzyme-linked immunosorbent assay (ELISA) versus double antigen ELISA for the detection of anti-infliximab antibodies. Inflamm. Bowel Dis. 18, 1628-1633 (2012).

[8] Wang, S.-L. et al. Development and validation of a homogeneous mobility shift assay for the measurement of infliximab and antibodies-to-infliximab levels in patient serum, J. Immunol. Methods 382, 177-488 (2012).

[9] Bendtzen, K. Immunogenicity of anti-TNF-α biotherapies: II. Clinical relevance of methods used for anti-drug antibody detection. B Cell Biol. 6, 109 (2015).

[10] Lallemand, C. et al. Reporter gene assay for the quantification of the activity and neutralizing antibody response to TNFα antagonists. J. Immunol Methods 373, 229-239 (2011).

[11] G. R. Gunn III et al., From the bench to clinical practice: understanding the challenges and uncertainties in immunogenicity testing for biopharmaceuticals. Clinical and Experimental Immunology, 184: 137-146 (2016).

[12] Ungar B, Chowers Y, Yavzori M, et al. The temporal evolution of antidrug antibodies in patients with inflammatory bowel disease treated with infliximab. Gut 2014; 63:1258-64.

Acknowledgement of the above references herein is not to be inferred as meaning that these are in any way relevant to the patentability of the presently disclosed subject matter.

### BACKGROUND OF THE INVENTION

The last decade had evidenced substantial evolution of therapy with the introduction of biologic agents aimed at specific components of the immune system. The major breakthrough was the introduction of anti-tumor necrosis factor alpha (TNFα) agents, namely Infliximab.

Therapeutic drug monitoring (TDM) of anti-TNF therapy has become the standard of care for many clinicians world-wide. Infliximab and adalimumab serum trough levels are positively associated with clinical response [1, 2]. Adequate trough levels were also associated with higher rates of mucosal healing and decreased incidence of long-term complications in both UC and CD [3-4].

The use of biologic drugs targeting TNFα, or any other biological target in IBD patients is often hampered by the appearance of anti-drug antibodies (ADA) which reduce the efficacy of the drug. Assessment of disease activity along with measurements of anti-TNF drug levels facilitates rational decisions on management of loss of response, optimization of disease control during maintenance therapy and possible cessation of treatment. Anti-drug antibody measurements aid in these clinical situations and are mostly useful in patients with loss of response for choosing the next step intervention [5].

The interference with drug activity could result from ADA-mediated increase of its clearance, or, in the case when ADA arise specifically against the drug's binding site thereby neutralizing its ability to bind the target, leading to clinical loss of response. Co-treatment with immunosuppressive agents can abrogate the appearance of antibodies, but is associated with significant side effects. Furthermore, it was recently shown that discriminating between neutralizing and non-neutralizing antibodies is of importance, and that detection of specific neutralizing antibodies, which compete for the target binding site is superior to the current antibody detection methods with respect to correlation with clinical loss of response and with the prediction of subsequent loss of response, at least in IBD patients receiving anti-TNFα treatment [6].

The current methods used for ADA detection in the clinic include a few variants of the bridging assay, relying on the bivalent structure of the antibodies and the anti-lambda chain based enzyme-linked immunosorbent assay (ELISA) using the lambda light chain of ADA for their detection [7]. Other methods, such as the homogenous mobility-shift assay (HMSA), use size-exclusion high-performance liquid chromatography (SE-HPLC) to quantitatively measure drug-antibody complexes in serum spiked with the labeled drug [8]. The limitation of these assays is the detection of any anti-drug binding activity without discrimination between neutralizing and non-neutralizing antibodies [9]. They are time consuming, laborious, sensitive to serum drug and as such, are not appropriate as a point of care assay.

Another type of ADA assay is the reporter gene assay [10]. This cell based assay, which does identify neutralizing antibodies, relies on activation of a TNFα-sensitive reporter gene. In the presence of active drug the reporter gene expression will decrease, while when neutralizing ADA are present in the serum, its expression will increase again. In this case, aside from requiring cell culture facilities and proficient laboratory personnel, an important limitation is that this assay is sensitive to excess drug in the serum as well.

G. R. Gunn III et al, review ELISA-based assays for assessing levels of neutralizing antibodies in patients treated with biological drugs [11].

Hence, effective and sensitive tools for timely prediction and monitoring of anti-drug immunogenicity are an unmet medical needs.

### SUMMARY OF THE INVENTION

According to a first aspect, the invention relates to methods for determining the level of neutralizing anti-drug antibodies (nADA) in a biological sample of a subject treated with a biological drug. In some embodiments, the method of the invention may comprise the following steps:

First, in step (a), incubating the biological sample with the biological drug immobilized directly or indirectly on a solid support. Step (b) involves providing the incubated sample of step (a) with a target of the biological drug, and incubating the target with the immobilized drug.

In step (c), determining the amount of the target bound to the immobilized drug. In some embodiments, determination of the amount of the target may be performed by detecting at least one detectable moiety associated either directly with the target or alternatively, indirectly, for example by detecting the bound target using a specific antibody associated either directly or indirectly with a detectable moiety. It should be noted that the amount of the labeled target determined, is indicative of the levels of neutralizing anti-drug antibodies present in the biological sample. In more specific embodiments, the levels of the neutralizing antibodies are in inverse correlation with the level of the detected target.

In a further aspect, the invention relates to prognostic method for evaluating, and/or assessing responsiveness of a subject to treatment with a biological drug, for monitoring disease progression and early prognosis of disease relapse. More specifically, such methods may comprise the following steps:

First, in step (a), determining the level of nADA in at least one biological sample of the subject, thereby obtaining an nADA value of the sample.

Next, in step (b), determining if the nADA value obtained in step (a) is any one of positive or negative with respect to a predetermined standard nADA value or to an nADA value in at least one control sample.

Step (c) involves classifying the subject as a non-responder or as a responder. More specifically, a positive nADA value of the sample, may indicate that the subject belongs to a pre-established population associated with non-responsiveness to the biological drug treatment. In other words, this indicates that the subject is a non-responder. However, a negative nADA value of the sample, may indicate that the subject belongs to a pre-established population associated with responsiveness to the biological drug treatment, thereby predicting, assessing and monitoring responsiveness of a subject to the treatment regimen. Specifically, that the subject is a responder.

In some embodiments, the level of nADA in at least one biological sample of the subject may be determined by the steps of: (a) incubating the biological sample with the biological drug immobilized directly or indirectly on a solid support; (b), providing the incubated sample of (a) with a target of said biological drug and incubating the target with the immobilized drug; and (c), determining the amount of the target bound to said immobilized drug. The amount is indicative of the levels of nADAs present in the biological sample, and in some embodiments, the amount is in inverse correlation with the level of the bound target.

In a further aspect, the invention relates to methods for determining the treatment regimen of a subject suffering from an immune-mediated disorder. The methods may comprise the steps of:

In a first step (a), determining the level of nADAs in at least one biological sample of the subject, thereby obtaining an nADA value of the sample.

In step (b), determining if the nADA value obtained in step (a) is any one of positive or negative with respect to a predetermined standard nADA value or to an nADA value in at least one control sample.

In step (c), determining treatment regimen for the subject, wherein:

(i) a positive nADA value of the sample, indicates that the subject belongs to a pre-established population associated with at least one of loss of response (LOR), inadequate response and intolerance to the biological drug treatment. the subject is recommended not to maintain the treatment. In other words, the subject is classified as a subject having, displaying or characterized in at least one of LOR, inadequate response and intolerance to the biological drug treatment. Alternatively, or additionally, the subject may be recommended to be administered with at least one immunosuppressive agent; and

(ii) a negative nADA value of the sample, indicates that the subject belongs to a pre-established population associated with responsiveness to the biological drug treatment. Specifically, a responsive subject. In some embodiments, the subject may be recommended to maintain the treatment.

In yet another aspect, the invention relates to a device for detecting nADAs in a biological sample of a subject treated with the biological drug. More specifically, the device of the invention may comprise the following elements or components:

In a first element (a), a labeling composition comprising a biological target of the biological drug. The target specifically recognizes and binds the biological drug. It should be noted that in some embodiments, the target may be associated, either directly or indirectly with at least one detectable moiety. In some alternative embodiments, the detection of the target may be accomplished using a specific antibody that is associated either directly or indirectly with a detectable moiety.

In a second element (b), the device of the invention may comprise a capture-composition comprising the biological drug immobilized directly or indirectly on a solid support; and

Finally, as a third element (c), the device may comprise a solid support suitable for the reception and transport of the biological sample.

Another aspect of the invention relates to a kit comprising:

(a), a biological drug immobilized directly or indirectly on a solid support;

(b), a biological target of the biological drug (optionally, associated with a detectable moiety); and optionally at least one of:

(c), instructions for use; (d), standard curves and/or or control samples; (e), at least one anti-lambda chain antibody (or alternatively, anti-kappa light chain antibodies), optionally associated with a second detectable moiety; and (f) at least one non-neutralizing antibody specific for said biological drug, wherein said non-neutralizing antibody is immobilized on a solid support.

In some embodiments, the kit of the invention may be suitable for predicting and assessing responsiveness of a subject to treatment with a biological drug, for monitoring disease progression and early prognosis of disease relapse.

In yet another aspect, the invention relates to methods for determining the level of an active biological drug in a biological sample of a subject treated with the biological drug. In some embodiments, the method may comprise the following steps:

In a first step (a), incubating the sample with at least one non-neutralizing antibody specific for said biological drug. The non-neutralizing antibody may be immobilized to a solid support.

In step (b), providing the incubated sample of step (a) with a target for the biological drug. In some embodiments, the target may be associated (either directly or indirectly) with a detectable moiety.

The next step (c), involves detecting the detectable moiety to determine the amount of the target. This amount may be indicative of the levels of the active drug present in the biological sample that is attached to the immobilized non-neutralizing antibody.

These and further aspects of the invention will become apparent by the hand of the following drawings.

### BRIEF DESCRIPTION OF THE FIGURES

In order to better understand the subject matter that is disclosed herein and to exemplify how it may be carried out in practice, embodiments will now be described, by way of non-limiting example only, with reference to the accompanying drawings, in which:

FIG. 1. A novel anti-drug neutralizing antibody assay

The biologic drug is first immobilized either directly or indirectly onto a solid matrix. ADAs-suspected serum is added, allowing anti-drug antibodies to bind the immobilized drug. After an optional washing step, a labeled form of the target is added and allowed to bind the immobilized drug. Excess unbound target is optionally washed off and bound target is measured. As shown in the lower panel of the figure, in the absence of neutralizing antibodies (A) the anti-antigen binding sites of the drug are free to bind the labeled target, while in the presence of neutralizing antibodies (B and C), and in contrast to non-neutralizing antibodies (D), the binding sites are blocked, preventing the target from binding to the drug and therefore a reduced signal is measured.

FIG. 2. TNFα binding in the presence of Infliximab neutralizing or non-neutralizing antibodies

Free Infliximab (white bar) or the indicated concentrations of neutralizing (gray bars) or non-neutralizing (dotted bars) antibodies were incubated for 30 min on Infliximab coated wells. Results are expressed as the percent of bound TNFα measured, compared to the baseline measurement obtained in the absence of antibody (black bar).

FIG. 3. Neutralization of TNFα binding to infliximab in the presence of sera

To ensure that the presence of sera does not interfere with the results, the assay was performed with pooled negative sera diluted 1:20 in 1% BSA in PBS. A standard ELISA plate was coated with 250 ng/ml Infliximab overnight and serial dilution (20 ng/ml to 2.5 ng/ml) of the neutralizing antibody was prepared in either 1% BSA in PBS or in 5% (1:20) pooled negative sera diluted in 1% BSA solution. It appears that the addition of sera does not affect the signal of bound TNT.

FIG. 4. Defining optimal serum concentration

The assay was performed as previously described, with the serial antibody dilution prepared in 2, 5 or 10% pooled negative sera diluted in 1% BSA in PBS.

FIG. 5. Measuring drug level utilizing labeled target as the readout and testing it in patients' sera

Commercial non-neutralizing anti-drug antibodies, are immobilized onto a solid matrix. Serum is then added, allowing the immobilized antibodies to capture the drug present in the sample. A labeled form of the target is added, binding to the captured active drug, thereby reflecting the amount of the active drug in the sample.

FIG. 6A-6B. Validation of an Infliximab sera level assay utilizing TNF for detection

FIG. 6A. Four parameter logistic regression model fitting the standard curve using Infliximab concentrations between 3.125 and 200 mg/ml. R²=0.9973.

FIG. 6B. Serum samples from 32 patients were evaluated for drug levels by the routine assay using anti-Fc for Infliximab detection and by the new assay. A high coefficient of correlation was found between the two methods.

### DETAILED DESCRIPTION OF THE INVENTION

Since the introduction of monoclonal antibodies for the treatment of immune mediated disorders such as IBD for example, the use of these agents has exponentially increased. Despite their proven and often clinically marked efficacy, biological agents are not immune to treatment failures, which can manifest as primary nonresponse, secondary loss of response or a failure to regain response after re-induction in a patient who has been previously exposed to the drug. Conversely, the substantial costs of these agents along with concerns about potential treatment-mediated adverse events, have led clinicians and some national health-payer agencies to consider cessation of these treatments after certain treatment goals are achieved, or to explore whether conventional dosing can be reduced in certain patients or clinical situations, such as in the postoperative setting. To meet these challenges, measuring levels of active drug and anti-drug antibodies, especially neutralizing anti-drug antibodies, which are elicited in a subset of patients, has emerged as a potentially powerful tool to elucidate mechanisms of loss of response and guide therapy in a sizable portion of patients. These measurements are then translated for choosing the optimal strategies for nonresponding patients and/or for tailoring continued therapy or even its cessation in patients who are doing well on maintenance therapy. Such a test based approach is an important leap towards individualized treatment of immune-mediated disorders such as IBD.

Therefore, the invention disclosed herein is of particular clinical relevance since in a first aspect, the invention relates to a method for determining the level of neutralizing anti-drug antibodies (nADAs) in a biological sample of a subject treated with a biological drug. In some embodiments, the method of the invention may comprise the following steps:

First, in step (a), incubating the biological sample with the biological drug immobilized directly or indirectly on a solid support. Step (b) involves providing the incubated sample of step (a) with a target of the biological drug, and incubating the target with the immobilized drug. It should be appreciated that the target used by the methods of the invention may be either associated (directly or indirectly) with a detectable moiety, or alternatively, a specific antibody or any other affinity molecule specific for said target (specifically, when bound to the immobilized drug), may be used for detecting the target. In yet some alternative embodiments, the target may not be associated directly or indirectly with a detectable moiety.

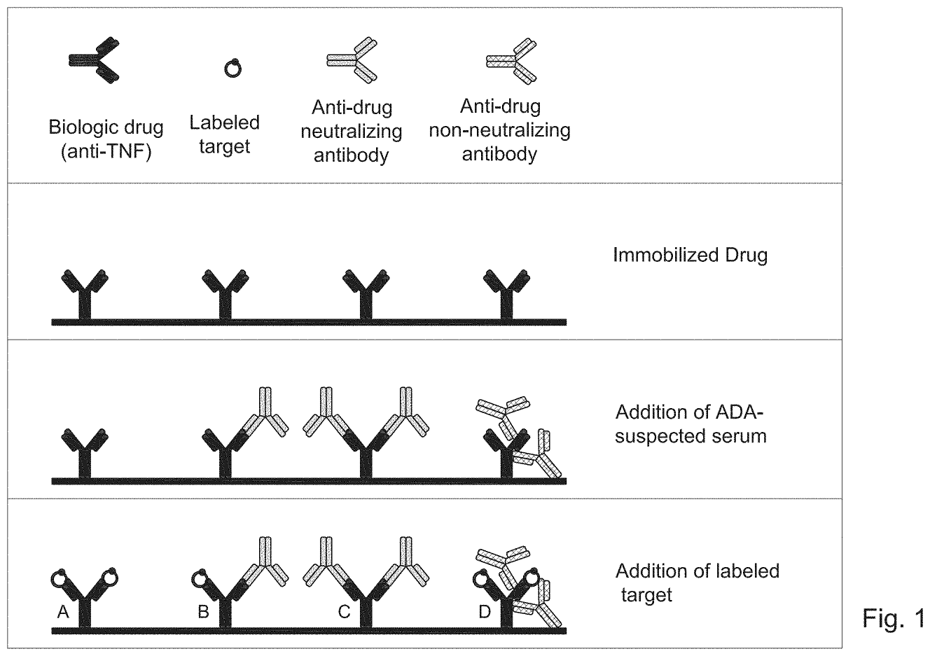

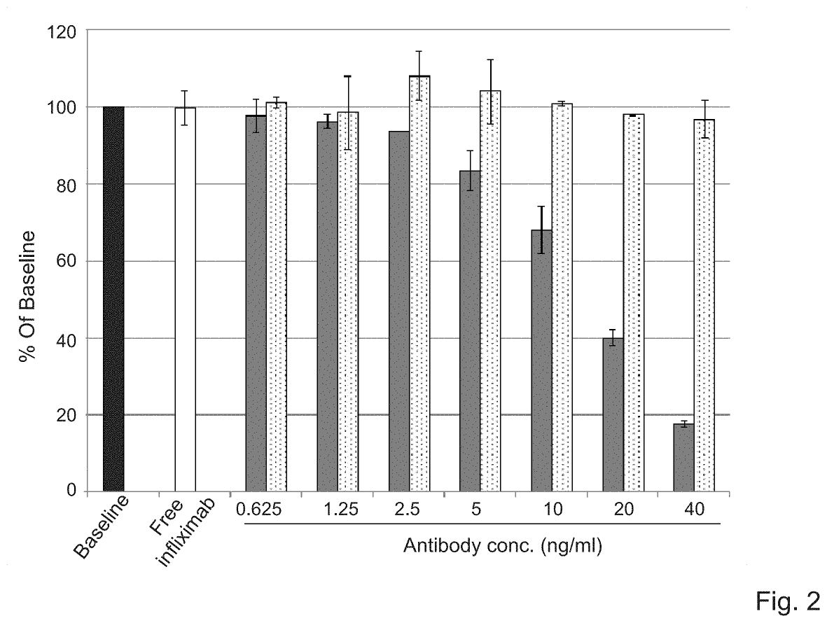

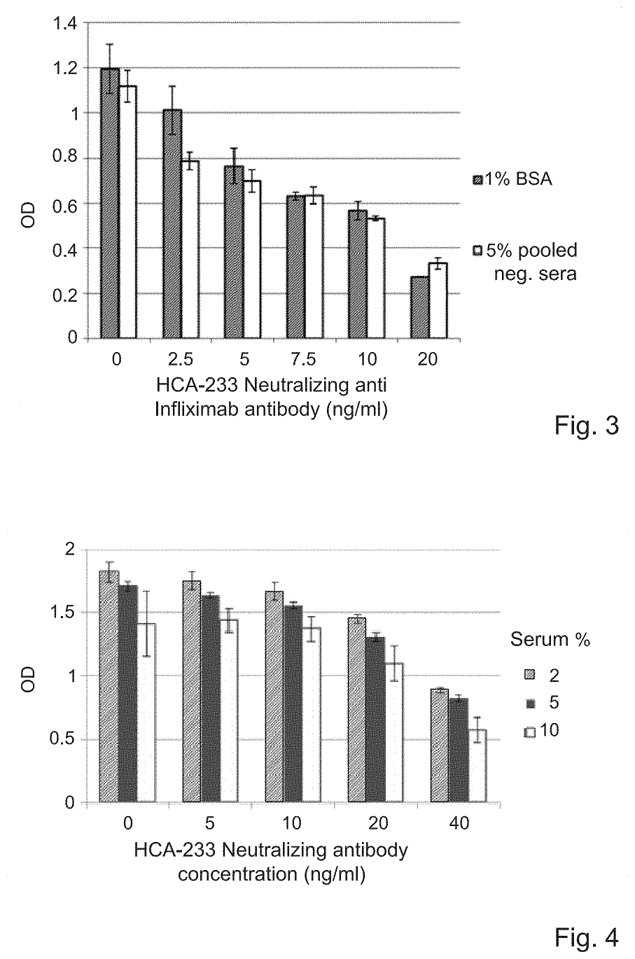

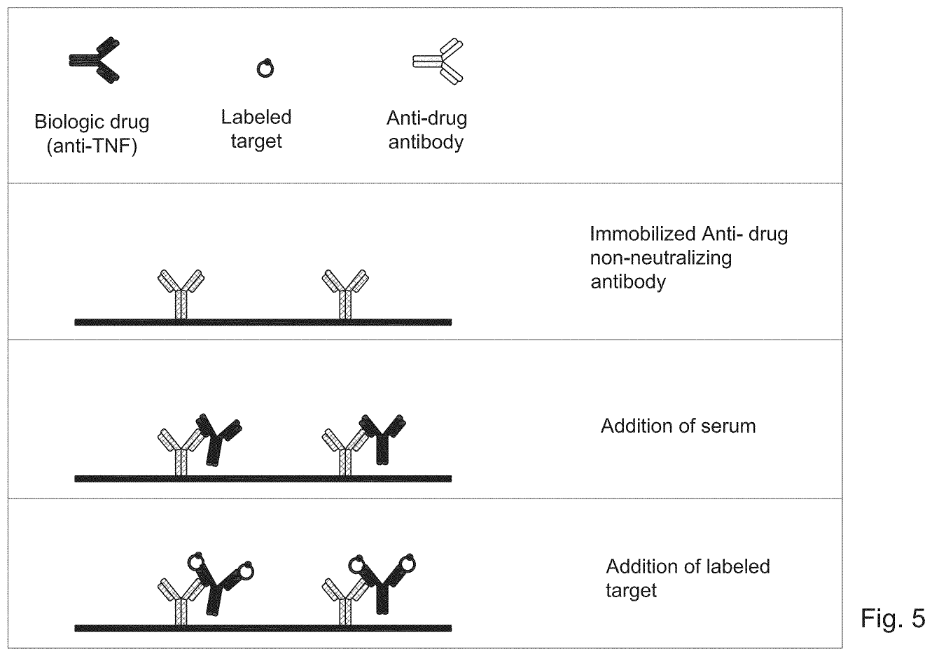

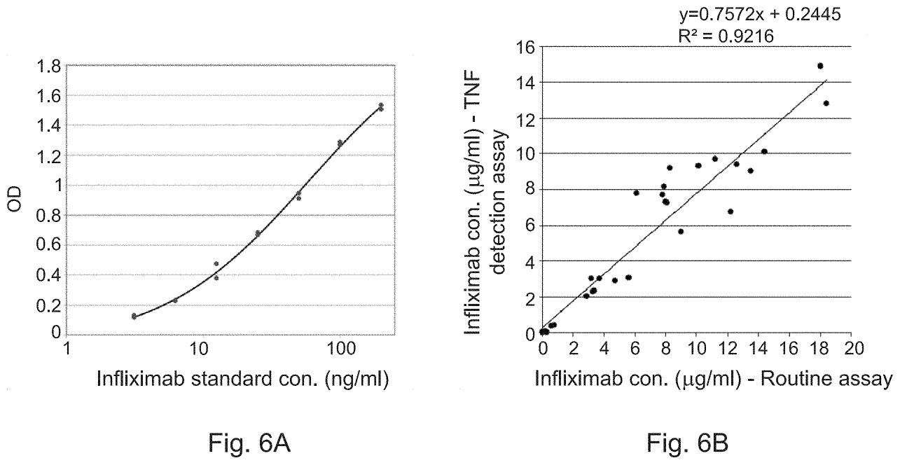

In step (c), determining the amount of the target bound to the immobilized drug. As indicated above, this step may be performed either by detecting the detectable moiety associated with the target, or alternatively, by using a specific antibody (or any affinity molecule) that recognizes and binds the target attached to the immobilized drug. It should be noted that the amount of the labeled target determined, is indicative of the levels of neutralizing anti-drug antibodies present in the biological sample. It should be noted that in certain embodiments, the levels or the amount of the nADAs in the sample may be in inverse correlation with the amount or level of the target bound to the immobilized drug. More specifically, high levels of the bound target indicate low levels of nADAs in the sample, and low binding of labeled target reflects high levels of nADAs in the sample that bind the immobilized drug thereby preventing binding of the target. In some embodiments, a non-limiting illustration of the method of the invention is presented by FIG. 1 and Example 1. Still further, in some particular and non-limiting embodiments, the level of the nADAs in the sample may reflect, or shade some indirect information that relates to the level of the active drug, where high levels of nADAs may usually reflect and indicated reduced active drug levels.

As used herein, the terms “drug”, “biological drug” and their plurals are used interchangeably and refer to drugs consisting of or comprising biological molecules or material, i.e. both, proteins, polypeptides, peptides, polynucleotides, oligonucleotides, polysaccharides, oligosaccharides and fragments thereof, as well as cells, tissues, biological fluids or extracts thereof, and which induce antibodies in a subject. In some embodiments, biological drugs may include proteins such as monoclonal antibodies, cytokines, soluble receptors, growth factors, hormones, enzymes, adhesion molecules and fusion proteins and peptides that are specific to certain targets known to modulate disease mechanisms. In yet some further embodiments, biological drugs may include or target any component participating in molecular and/or cellular processes such as, cell cycle, cell survival, apoptosis, immunity and the like. In more specific embodiments, biological drugs may be any checkpoint protein's or any modulators or inhibitors thereof, or any combinations thereof. In yet some further embodiments, biological drugs (or their precursors or components) may be isolated from living sources human, animal, plant, fungal, or microbial.

Still further in some embodiments, “biological drug” or “biologics” refers to a class of therapeutics that are produced by means of biological processes involving recombinant DNA technology which are usually one of three types: (a) substances that are similar to the natural occurring proteins: (b) monoclonal antibodies; and (c) receptor constructs or fusion proteins, usually based on a naturally occurring receptor linked to the immunoglobulin frame. Major kinds of biologics include but are not limited to: Blood factors (such as Factor VIII and Factor IX), Thrombolytic agents (such as tissue plasminogen activator), Hormones (such as insulin, glucagon, growth hormone, gonadotrophins), Haematopoietic growth factors (such as Erythropoietin, colony stimulating factors), Interferons (such as interferons-α, -β, -γ), Interleukin-based products (such as Interleukin-2), Vaccines (such as Hepatitis B surface antigen) and monoclonal antibodies. Non-limiting examples of biological drugs made with recombinant DNA technology may include at least one of: abatacept (Orencia®), that is a fusion protein composed of the Fc region of the immunoglobulin IgG1 fused to the extracellular domain of CTLA-4, used to treat autoimmune diseases like rheumatoid arthritis, by interfering with the immune activity of T cells; erythropoietin or Epoetin alfa (Epogen®), that is a human erythropoietin produced in cell culture using recombinant DNA technology, that stimulates erythropoiesis and is used to treat anemia, commonly associated with chronic renal failure and cancer chemotherapy; Muromonab-CD3 (Orthoclone OKT3®), that is a monoclonal antibody working as an immunosuppressant drug given to reduce acute rejection in patients with organ transplants. It binds to the T cell receptor-CD3-complex on the surface of circulating T cells thereby inducing blockage and apoptosis of the T cells; Abcixinmab (ReoPro®), that is a glycoprotein IIb/IIIa receptor antagonist mainly used during and after coronary artery procedures; Basiliximab (Simulect®), that is a chimeric CD25 monoclonal antibody of the IgG1 isotype, used as an immunosuppressant to prevent immediate transplant rejection; and Palivizumab (Synagis®), that is a humanized monoclonal antibody (IgG) directed against an epitope in the A antigenic site of the F protein of the respiratory syncytial virus (RSV).

As detailed above, biological drug are known in some instances to elicit formation in vivo of Anti-drug antibodies (ADAs) and their detection has generally been equated as a measure of immunogenicity. Most adverse effects consequential to ADA formation, such as pharmacological abrogation, impact on therapeutic exposure, or hypersensitivity reactions, are a consequence of formation of immune complexes between the ADA and therapeutic protein. Their levels, kinetics of interaction, size, polyclonal diversity, distribution, and Fc-mediated physiological effects can be potentially translated to clinically observable adverse effects. ADAs represent a very complex set of analytes, as they are usually polyclonal, may include different isotypes [immunoglobulin (Ig)G, IgA, IgM or IgE], bind to different regions (‘domains’) of the drug molecule, vary in affinity (binding strength) and can differ between patients. It should be appreciated that nADAs as specified herein, may be also applicable in any other aspect of the invention disclosed herein after. There are two main types of ADA: Neutralizing antibodies (NAb) and non-neutralizing antibodies (non-NAb). Neutralizing antibodies (NAb) are a subset of binding ADA that bind to the drug and inhibit its pharmacological function by preventing target binding. Accordingly, non-neutralizing antibodies (non-NAb) are ADA that bind to sites on the drug molecule that do not affect target binding and thereby usually do not impact the drug's pharmacodynamic activity. Once the ADA (‘binding’ ADA) is detected, it is useful to determine their neutralizing ability, particularly for drugs with short half-lives (minutes to a few days) or those with an identical, endogenous counterpart.

NAbs can inhibit drug activity soon after the drug is administered, but non-NAb do not inhibit the pharmacodynamic activity of the drug. Therefore, the method of the invention is of particular clinical interest since it enables detection of Neutralizing antibodies (NAb), and as such, may in some embodiments, allow the evaluation of the active biological drug, and moreover, the potential of the treated patient to respond to the biological treatment. More specifically, the amount of the neutralizing antibodies that may inhibit the desired activity of the biological drug on the desired target. Neutralizing antibodies, in this connection may inhibit, reduce, prevent or eliminate the activity of the biological drug, e.g., binding of the biological drug to its target and thereby the activity associated therewith, in about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% or more, as compared to the activity of the biological drug in the absence of nADAs.

In some embodiments, step (a) of the method of the invention may be performed under conditions suitable for recognition and binding of the drug-target, or alternatively or additionally, conditions suitable for binding of the nADAs in the sample to the immobilized drug. In yet some further embodiments, this step may be followed by washing step or at least removal of the sample. Washing steps, may in some embodiments involve the use of any suitable washing buffer that is stringent sufficiently to remove most of the non-specific binding, but also retains only the specific binding of the labeled target to the immobilized biological drug. Such optional washing step may be performed one time or more, for example, 1, 2, 3, 4, 5, 6, 7, 8, 9, 10 times or more if required. In some alternative or optional embodiments, the methods of the invention may further comprise an additional dissociation step. In some embodiments, such dissociation step may be performed prior to step (a). As used herein, the term dissociation step relates to a pretreatment step applied to the biological sample prior to incubation of step (a), performed in conditions suitable for releasing and/or dissociating any complexes that may interfere with the performance or accuracy of the test. In some specific embodiments, such dissociation step may release or dissociate drug/anti-drug antibody complexes, thereby facilitating binding of the nADAs to the immobilized drug.

In some particular and non-limiting embodiments the dissociation step may involve pretreating the samples for about 1 to 30 minutes, specifically, 1, 2, 3, 4, 5, 6, 7, 8, 9. 10, 11, 12, 13, 14, 15, 16, 17, 18, 19, 20, 21, 22, 23, 24, 25, 26, 27, 28, 29, 30 or more minutes, more specifically, 15 minutes with at least one dissociating agent. Non limiting examples for an appropriate dissociation agent include any acidic substance, for example, any acid such as Acetic acid, Glycine-HCl or any equivalent acid, followed by a neutralizing buffer. In some particular embodiments the acid used as a dissociating agent may be present in an amount of between about 10 mM to about 1000 mM or more. More specifically, 10, 20, 30, 40, 50, 60, 70, 80, 90, 100, 150, 200, 250, 300, 350, 400, 450, 500, 550, 600, 650, 700, 750, 800, 850, 900, 950, 1000 mM or more. In yet some further specific embodiments, the dissociating agent used may be acetic acid in an amount of between about 300 to 600 mm. In yet some further specific embodiments, the acetic acid used may be in an amount of 300 mM. Still in some further embodiments, Glycine-HCl may be used as a dissociating agent. In certain specific embodiments an amount of 100 mM Glycine-HCl may be used. As indicated above, following the dissociation step, the dissociating agent may be neutralized by the addition of a neutral buffer such as Tris 1M.

In some embodiments, the biological drug may be immobilized directly or indirectly on the solid support (also called coating step) at different concentrations. In more specific embodiments, such drug concentration may range from between about 1 ng/ml to about 10000 ng/ml, specifically, about 1, 5, 10, 15, 20, 25, 30, 35, 40, 45, 50, 55, 60, 65, 70, 75, 80, 85, 90, 95, 100 ng/ml or more, specifically, about 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 ng/ml, or more, specifically, 550, 600, 650, 700, 750, 800, 850, 900, 950, 1000 ng/ml, or more, specifically, 2000, 1500, 3000, 3500, 4000, 4500, 5000, 5500, 6000, 6500, 7000, 7500, 8000, 8500, 9000, 9500, 10000 ng/ml, or even more. In yet some further specific embodiments, the immobilized biological drug may be in an amount ranging from 100 ng/ml to 500 ng/ml. In more specific embodiments, the biological drug concentration may be 250 ng/ml. Still further, the next step of the method of the invention (b), involves providing the incubated sample of (a) with a target of the biological drug and incubating the target with the immobilized drug. In certain embodiments, determination of the level of the labeled target in step (c) by detecting its detectable moiety, may further involve detecting by any suitable means, a signal from the detectable moiety of the labeled target that correlates with the level of the labeled target bound to the immobilized drug. The amount of the labeled target bound to the immobilized drug, correlates (e.g., inverse correlation) with the amount of the neutralizing ADA in the sample of the subject. According to some embodiments, the signal detected from the sample by any one of the experimental methods detailed herein below correlates to the amount of bound target and thus reflects the amount of neutralizing ADA. It should be noted that in certain embodiments, such signal-to-level data may be calculated and derived from a standard curve.

Thus, in certain embodiments, the method of the invention may optionally further involve the use of a standard curve created by detecting a signal for each one of increasing pre-determined concentrations of the labeled biological target, which is indicative with the level of neutralizing ADA in the biological sample. Obtaining such a standard curve may be indicative to evaluate the range at which the levels of detected labeled bound target correlate inversely with the concentrations of the neutralizing ADA present in the biological sample. It should be noted in this connection that at times when no change in the level of detected labeled target is observed, the standard curve should be evaluated in order to rule out the possibility that the measured level is not exhibiting a saturation type curve, namely a range at which increasing concentrations exhibit the same signal.

It must be appreciated that in certain embodiments such standard curve as described above may be also part or component in any of the kits provided by the invention as described herein after. As described herein, the methods of the invention, as well as the devices and kits disclosed herein after, disclose that the biological drug may be immobilized directly or indirectly on a solid support. As used herein, the term “immobilized” refers to a stable association of the biological drug (or non-neutralizing antibody) with a surface of a solid support. By “stable association” is meant a physical association between two entities in which the mean half-life of association is one day or more, two days or more, one week or more, one month or more, including six months or more e.g., under physiological conditions. According to certain embodiments, the stable association arises from a covalent bond between the two entities, a non-covalent bond between the two entities (e.g., an ionic or metallic bond), or other forms of chemical attraction, such as hydrogen bonding, Van der Waals forces, and the like. Solid support suitable for use in the methods, devices and kits of the present invention is typically substantially insoluble in liquid phases. Solid supports of the current invention are not limited to a specific type of support. Rather, a large number of supports are available and are known to one of ordinary skill in the art. Thus, useful solid supports include solid and semi-solid matrixes, such as aerogels and hydrogels, resins, beads, biochips (including thin film coated biochips), microfluidic chip, a silicon chip, nanoparticles, polymers, multi-well plates (also referred to as microtiter plates or microplates), membranes, filters, conducting and non-conducting metals, glass (including microscope slides) and magnetic supports. More specific examples of useful solid supports include, silica gels, polymeric membranes such as nitrocellulose, particles, derivatized plastic films, glass beads, cotton, plastic beads, alumina gels, polysaccharides such as Sepharose, nylon, latex bead, magnetic bead, paramagnetic bead, superparamagnetic bead, starch and the like. In yet some further embodiments, in case electrochemical assays are applied by the methods, devices and kits of the invention, solid support may further include nano- and micro-sized materials, such as gold nanoparticles (GNPs), carbon nanotubes (CNTs), graphene (GR), magnetic particles (MBs), quantum dots (QDs) and conductive polymers. In yet some further embodiments, such nano- and micro-sized materials, used as a solid support may be employed to modify an electrode surface. Thus, in some embodiments, particularly when electrochemical assays are applied by the invention, the solid support may be either comprising or connected directly or indirectly to conductive material, such as electrode or any other modified electric surface that may be suitable for transducing an electrochemical signal thrilled by the recognition and binding of the immobilized drug and its target. More specifically, such electrode surface enables the electron transfer from the label (detectable moiety) to the electrode, and is affected by the binding event which occurs on the electrode surface. In yet some further embodiments, electrodes suitable for such use may include glassy carbon electrodes that may be further modified by the solid support, and screen printed electrodes (SPE).

As described in the examples, the method of the invention may be particularly suitable, in some embodiments thereof, for the detection of nADAs elicited in a subject treated with an antibody. Therefore, in some embodiments, the suitable biological drug for the method of the invention may be an antibody directed against a biological target.

The term “antibody” as used herein, means any antigen-binding molecule or molecular complex that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region (CH). The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Typically, an antibody is composed of two immunoglobulin (Ig) heavy chains and two Ig light chains. In humans, antibodies are encoded by three independent gene loci, namely kappa (κ) chain (Igκ) and lambda (λ) chain (Igλ) genes for the Light chains and IgH genes for the Heavy chains, which are located on chromosome 2, chromosome 22, and chromosome 14, respectively. The antibody used by the method of the invention may be any one of a polyclonal, a monoclonal or humanized antibody or any antigen-binding fragment thereof. The term “an antigen-binding fragment” refers to any portion of an antibody that retains binding to the antigen. Examples of antibody functional fragments include, but are not limited to, complete antibody molecules, antibody fragments, such as Fv, single chain Fv (scFv), complementarity determining regions (CDRs), V_{L} (light chain variable region), V_{H} (heavy chain variable region), Fab, F(ab)₂′ and any combination of those or any other functional portion of an immunoglobulin peptide capable of binding to target antigen.

As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries. The term antibody also includes bivalent molecules, diabodies, triabodies, and tetrabodies.

References to “V_{H}” or a “VH” refer to the variable region of an immunoglobulin heavy chain, including an Fv, scFv, a disulfilde-stabilized Fv (dsFv) or Fab. References to “V_{L}” or a “VL” refer to the variable region of an immunoglobulin light chain, including of an Fv, scFv, dsFv or Fab. More specifically, the phrase “single chain Fv” or “scFv” refers to an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. A single chain antibody applicable for the invention, e.g., may bind as a monomer. Other exemplary single chain antibodies may form diabodies, triabodies, and tetrabodies.

It should be appreciated that in some embodiments, any antibody used by the methods, devices and kits of the invention as a biological drug or as non-neutralizing antibody, is not a naturally occurring antibody. Specifically, any of the antibodies used herein cannot be considered as a product of nature. In yet some further embodiments, immobilization of any of the antibodies used to create an immobilized drug or immobilized non-neutralizing antibody, clearly distinguishes the product used from its natural counterpart.

In some embodiment wherein the biological drug of the method of the invention is an antibody, the biological target provided in step (b) therefore represents and comprise an epitope. The terra “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

It should be appreciated that antibodies and antigens as specified herein, may be also applicable in any other aspect of the invention disclosed herein after.

Furthermore, in certain embodiments, the biological target of the biological drug used by the method of the invention may be a cytokine.

The term “cytokine” generally refers to proteins produced by a wide variety of hematopoietic and non-hematopoietic cells that affect the behavior of other cells. They act through receptors, and are especially important in the immune system; cytokines modulate the balance between humoral and cell-based immune responses, and regulate the maturation, growth, and responsiveness of particular cell populations. Their particular importance in the regulation of the immune response motivated the production of biological drug to specifically target them. Cytokines may be such as Acylation stimulating protein, Adipokine, Albinterferon, CCL1, CCL2, CCL3, CCL5, CCL6, CCL7, CCL8, CCL9, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, Cerberus, protein, Chemokine, Colony-stimulating factor, CX3CL1, CX3CR1, CXCL1, CXCL2, CXCL3, CXCL5, CXCL6, CXCL7, CXCL9, CXCL10, CXCL11, CXCL13, CXCL14, CXCL15, CXCL16, CXCL17, Erythropoietin, FMS-like tyrosine kinase 3 ligand, GcMAF, Granulocyte colony-stimulating factor (or CSF 3), Granulocyte macrophage colony-stimulating factor (or CSF2), IL 17 family, IL-10 family, Interferon, Interferon beta-1a, Interferon beta-1b, Interferon gamma, Interferon type I, Interferon type II, Interferon type III, Interferon-stimulated gene, Interleukin 1 receptor antagonist, Interleukin 8, Interleukin 12, Interleukin-18, Leukemia inhibitory factor, Leukocyte-promoting factor, Lymphokine, Lymphotoxin, Lymphotoxin alpha, Lymphotoxin beta, Macrophage colony-stimulating factor (CSF1), Macrophage inflammatory protein, Macrophage-activating factor, Monokine, Myokine, Myonectin, Nicotinamide phosphoribosyltransferase (NAmPRTase or Nampt) also known as pre-B-cell colony-enhancing factor 1 (PBEF1), Oncostatin M, Oprelvekin, Platelet factor 4, Receptor activator of nuclear factor kappa-B ligand (RANKL), also known as tumor necrosis factor ligand superfamily member 11 (TNFSF11), stromal cell-derived factor 1 (SDF1), also known as C—X—C motif chemokine 12 (CXCL12), tumor necrosis factor (TNF) superfamily such as Tumor necrosis factor alpha, Lymphotoxin-alpha, T cell antigen gp39 (CD40L), CD27L, CD30L, FAST-, 4-1BBL, OX40L, TNF-related apoptosis inducing ligand (TRAIL), Vascular endothelial growth inhibitor (VEGI), also known as TNF-like ligand 1A (TL1A), XCL1, XCL2, XCR1. It should be appreciated that cytokines as specified herein, may be also applicable in any other aspect of the invention disclosed herein after.

More specifically, in some embodiments, tumor necrosis factor (TNF, tumor necrosis factor alpha, TNFα, cachexin, or cachectin) is a cytokine of particular interest. It is involved in systemic inflammation and is one of the cytokines that make up the acute phase reaction. It is produced chiefly by activated macrophages, although it can be produced by many other cell types such as CD4+ lymphocytes, NK cells, neutrophils, mast cells, eosinophils, and neurons.

In some specific embodiments the biological target may be a cytokine. More specifically, at least one cytokine of particular interest in the present invention may be tumor necrosis factor alpha (TNFα). In more specific embodiments, the biological target may be human TNFα. In yet some further embodiments, TNFα may comprise the amino acid sequence as denoted by accession number NP_000585.2. In yet some further embodiments, a biological target used by the present invention may be the human TNFα that comprises the amino acid sequence as denoted by SEQ ID NO. 1. In yet some further embodiments, such human TNFα may be encoded by the nucleic acid sequence as denoted by SEQ ID NO. 2. Biological activities attributed to TNF-α include induction of pro-inflammatory cytokines (such as interleukins IL-1 and IL-6), enhancement of leukocyte movement or migration from the blood vessels into the tissues (by increasing the permeability of endothelial layer of blood vessels), and increasing the release of adhesion molecules. It should be noted that in some embodiments, a biological drug directed at TNF-α (that is served as a target to such drug), that may be used by the invention, may block and inhibit at least one of said TNF-α activities disclosed herein. Thus, in yet some further embodiments, nADAs detected by the methods of the invention may be any antibodies that prevent the blocking effect of the biological drug on TNF-α activities discussed herein.

Therefore, in some further embodiments, where the target used by the method of the invention may be at least one cytokine, specifically, TNFα, the drug may be an antibody specific for TNFα. More specifically, the drug may be a monoclonal antibody specific for TNFα. Non-limiting examples for such antibody that may be used in the methods of the invention include at least one of infliximab, etanercept, adalimumab, certolizumab pegol, golimumab, any biosimilar thereof and any combinations of the same.

In more specific embodiments, such biosimilar may include but are not restricted to, Remsima/INFLECTRA® (infliximab-dyyb), SB4 etanercept, SB2 infliximab and SB5 adalimumab.

TNF inhibitors are pharmaceutical drugs that suppresses the physiologic response to tumor necrosis factor (TNF), which is part of the inflammatory response. Inhibition of TNF effects can be achieved using a monoclonal antibody such as infliximab REMICADE®, etanercept, ENBREL®, adalimumab HUMIRA®, certolizumab pegol CIMZIA®, golimumab, SIMPONI®, and any biosimilars thereof, to name but a few, Remsimal/INFLECTRA® (infliximab-dyyb), SB4 etanercept, SB2 infliximab and SB5 adalimumab. Thalidomide (Immunoprin) and its derivatives lenalidomide (Revlimid) and pomalidomide (Pomalyst, Imnovid) are also active against TNF.

In some specific embodiments, the biological drug used by the methods of the invention may be infliximab. The term “infliximab” refers to the anti-TNF antibody marketed as REMICADE®, having FDA Unique Ingredient Identifier (UNII): B72HH48FLU and DRUG BANK Accession number DB00065. It is an Immunoglobulin G, (human-mouse monoclonal cA2 heavy chain), disulfide with human-mouse monoclonal cA2 light chain, dimer. More specifically, Infliximab is used to treat immune-mediated diseases such as Crohn's disease, ulcerative colitis, psoriasis, psoriatic arthritis, ankylosing spondylitis, and rheumatoid arthritis as well as Behcet's disease and other conditions. Infliximab is administered by intravenous infusion, typically at six- to eight-week intervals, but cannot be given orally.

Infliximab is a purified, recombinant DNA-derived chimeric human-mouse IgG monoclonal antibody that consists of mouse heavy and light chain variable regions combined with human heavy and light chain constant regions. It has a serum half-life of 9.5 days and can be detected in serum 8 weeks after infusion treatment.

Infliximab neutralizes the biological activity of TNF-α by binding with high affinity to both the soluble and transmembranal forms of TNF-α thereby inhibiting the effective binding of TNF-α with its receptors.

Infliximab has high specificity for TNF-α, and does not neutralize TNF beta (TNFβ, also called lymphotoxin α), an unrelated cytokine that uses different receptors from TNF-α. Blocked actions of TNF-α further leads to downregulation of local and systemic pro-inflammatory cytokines (i.e. IL-1, IL-6), reduction of lymphocyte and leukocyte migration to sites of inflammation, induction of apoptosis of TNF-producing cells (i.e. activated monocytes and T lymphocytes), increased levels of nuclear factor-κB inhibitor, and reduction of reduction of endothelial adhesion molecules and acute phase proteins. Infliximab also attenuates the production of tissue degrading enzymes synthesized by synoviocytes and/or chondrocytes.

In yet some further specific embodiments, the biological drug used by the methods of the invention may be etanercept. The term “etanercept” refers to the anti-TNF antibody marketed as ENBREL®, having FDA Unique Ingredient Identifier (UNII): OP401G7OJC and DRUG BANK Accession number DB00005. Etanercept is a fusion protein produced by recombinant DNA. It fuses the TNF receptor to the constant end of the IgG1 antibody as follows: residues 1-235-are of Tumor necrosis factor receptor (human) fusion protein with residues 236-467-immunoglobulin G1 (human γ1-chain Fc fragment). It is a large molecule, with a molecular weight of 150 kDa.

In still further specific embodiments, the biological drug used by the methods of the invention may be adalimumab, The terms “adalimumab” refers to the anti-TNF antibody marketed as HUMIRA®, having FDA Unique Ingredient Identifier (UNIT): FYS6T7F842 and DRUG BANK Accession number DB00051. It is an immunoglobulin G1, (human monoclonal D2E7 heavy chain), disulfide with human monoclonal D2E7 light chain, dimer.

In yet some further specific embodiments, the biological drug used by the methods of the invention may be certolizumab pegol. The term “certolizumab pegol” refers to the anti-TNF antibody marketed as CIMZIA®, having FDA Unique Ingredient identifier (UNII): UMD07X179E. It is a polyethylene-glycolated Fab′ fragment of TUMOR NECROSIS FACTOR antibody that binds specifically to TNFα and neutralizes it in a dose-dependent manner.

In some further specific embodiments, the biological drug used by the methods of the invention may be golimumab. The term “golimumab” refers to the anti-TNF antibody marketed as SIMPONI®, having FDA Unique Ingredient Identifier (UNII): 91X1KLU43E. It is an Immunoglobulin G1, (human monoclonal CNTO 148 gamma1-chain), disulfide with human monoclonal CNTO 148 kappa-chain, dimer. Its molecular weight is approximately 147 kDa.

In still further specific embodiments, the biological drug used by the methods of the invention may be Ustekinumab. The term “Ustekinumab” refers to a humanized monoclonal antibody that binds to IL-12 and IL-23 marketed as STELARA®, having FDA Unique Ingredient Identifier (UNII): FU77B4U5Z0. It is an immunoglobulin G1, anti-(human interleukin 12 p40 subunit) (human monoclonal CNTO 1275 gamma1-chain), disulfide with human monoclonal CNTO 1275 kappa-chain, dimer. It should be appreciated that in certain embodiment, the drug target used by the methods of the invention may be any biosimilar, specifically, any approved biosimilar of the aforementioned originator biologics.

In still further specific embodiments, the biological drug used by the methods of the invention may be Etrolizumab. The term “Etrolizumab” or “rhuMAb Beta7” refers to a humanized monoclonal antibody against the β7 subunit of integrins α4β7 and αEβ7, having FDA Unique Ingredient Identifier (UNII): I2A72G2V3J. It is an Immunoglobulin G1, anti-(human integrin alpha47/integrin alphaE7) (human-rat monoclonal rhuMAb Beta7 heavy chain), disulfide with human-rat monoclonal rhuMAb Beta7 light chain, dimer. It should be appreciated that in certain embodiment, any biosimilar of the above, specifically, any approved biosimilar, may be used by the methods of the invention as a target. In yet some further embodiments, the drug used by the methods of the invention may be Mirikizumab (LY3074828) that targets interleukin 23A and is in clinical use in treating inflammatory conditions such as Moderate-to-Severe Ultracerative Colitis, In yet some further embodiments the methods of the invention may use Risankizumab (ABBV-066) that is an anti-IL23 antibody being clinically used for the treatment of multiple inflammatory diseases, including psoriasis, Crohn's disease and psoriatic arthritis.

In more specific embodiments, the biosimilar may be any approved biosimilar of the aforementioned originator biologics.

The term “biosimilar” means a biological product that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. For example, if the reference anti-TNF monoclonal antibody is infliximab, an anti-TNF biosimilar monoclonal antibody approved by drug regulatory authorities with reference to infliximab is a “biosimilar to” infliximab or is a “biosimilar thereof” of infliximab.

In Europe, a similar biological or “biosimilar” medicine is a biological medicine that is similar to another biological medicine that has already been authorized for use by the European Medicines Agency (EMA.). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA. A biosimilar as described herein may be similar to the reference medicinal product by way of quality characteristics, biological activity, mechanism of action, safety profiles and/or efficacy, or any combinations thereof. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies.

As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97 percent or greater to the amino acid sequence of its reference medicinal product, e.g., 97 percent, 98 percent, 99 percent or 100 percent. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

In some specific embodiments, the aforementioned biological drugs have been developed for the treatment of immune-mediated disorder, such as Inflammatory bowel disease (IBD). In yet some other specific embodiments, the methods of the invention may be applicable for subject suffering from an immune-mediated disorder.

An “Immune-related disorder” or “ Immune-mediated disorder”, as used herein encompasses any condition that is associated with the immune system of a subject, either through activation or inhibition of the immune system, or that can be treated, prevented or diagnosed by targeting a certain component of the immune response in a subject, such as the adaptive or innate immune response. The immune-related disorder may be a chronic inflammatory condition, specifically, any one of an inflammatory disease, viral infections, an autoimmune disease, metabolic disorders and a proliferative disorder, specifically, cancer. In some embodiments, an immune-mediated disorder may be at least one of inflammatory disease, an autoimmune disease and a proliferative disorder (specifically, cancer). Thus, in more specific embodiments, the methods of the invention are suitable for at least one of inflammatory disorder, an autoimmune disease and a proliferative disease.

The general term “inflammatory disorder” relates to disorders where an inflammation is a main response to harmful stimuli, such as pathogens, damaged cells, or irritants. Inflammation is a protective response that involves immune cells, blood vessels, and molecular mediators, as well as the end result of long-term oxidative stress.

“Inflammatory disorders” are a large group of disorders that underlie a vast variety of human diseases. Also, the immune system can be involved in inflammatory disorders, stemming from abnormal immune response of the organism against substances of its own, or initiation of the inflammatory process for unknown reason, i.e. autoimmune and auto-inflammatory disorders, respectively. Non-immune diseases with etiological origins in inflammatory processes include cancer, atherosclerosis, and ischemic heart disease.

The purpose of inflammation is to eliminate the initial cause of cell injury, clear out necrotic cells and tissues and to initiate tissue repair. The classical physiological signs of acute inflammation are pain, heat, redness, swelling, and loss of function. A series of biochemical events propagates and matures the inflammatory response, involving the local vascular system, the immune system, and various cells within the injured tissue. Prolonged inflammation, known as “chronic inflammation”, leads to a progressive shift in the type of cells present at the site of inflammation and is characterized by simultaneous destruction and healing of the tissue from the inflammatory process. Inflammation also induces high systemic levels of specific cytokines designated as pro-inflammatory cytokines which include IL-1α, IL-6, IL-8, IFN-γ, TNF-α, IL-17 and IL-18. The inflammatory response must be actively terminated when no longer needed to prevent unnecessary “bystander” damage to tissues. Failure to do so results in chronic inflammation, and cellular destruction.

The term “pathological conditions associated with inflammation” as used herein relates to at least one but not limited to the following: inflammatory bowel disease (e.g., Crohn's disease, ulcerative colitis), arthritis (ankylosing spondylitis, systemic lupus erythematosus, rheumatoid arthritis, psoriatic arthritis), asthma, atherosclerosis, dermatitis and psoriasis.

In more specific embodiments, the immune-mediated disorder related to the method of the invention may be inflammatory bowel disease (IBD).

Inflammatory bowel diseases (IBD) are common gastrointestinal disorders, that can be perceived as being the result of inappropriate activation of the mucosal immune system leading to intestinal damage and associated extra intestinal manifestations. IBD is a group of inflammatory conditions of the colon and small intestine. The major types of IBD are Crohn's disease and ulcerative colitis (UC). Other forms of IBD account for far fewer cases. These are collagenous colitis, lymphocytic colitis, ischemic colitis, diversion colitis, and indeterminate colitis, in cases where it is impossible to make a definitive diagnosis distinguishing Crohn's disease from ulcerative colitis.

The main difference between Crohn's disease and UC is the location and nature of the inflammatory changes. Crohn's disease can affect any part of the gastrointestinal tract, from mouth to anus (skip lesions), although a majority of the cases start in the terminal ileum. Ulcerative colitis, in contrast, is restricted to the colon and the rectum. Microscopically, ulcerative colitis is restricted to the mucosa (epithelial lining of the gut), while Crohn's disease affects the whole bowel wall. Finally, Crohn's disease and ulcerative colitis present with extra-intestinal manifestations (such as liver problems, arthritis, skin manifestations and eye problems) in different proportions. Crohn's disease and ulcerative colitis share the same symptoms such as diarrhea, vomiting, weight loss, fever and abdominal pain.

A recent hypothesis posits that IBD may be caused by an over-active immune system attacking various tissues of the digestive tract, because of the lack of traditional targets such as parasites and worms.

There are several extra-intestinal manifestations that accompany IBD, for example: autoimmune phenomena, wherein immune complexes have a role in target organ damage. Patients with IBD (UC only) have antibodies against components of colon cells and several different bacterial antigens (mainly CD). These antigens are supposed to gain access to the immune system as a consequence of epithelial damage.

In some specific embodiments, the immune-mediated disorder applicable for the diagnostic and prognostic methods of the invention may be inflammatory bowel disease, specifically, any one of ulcerative colitis (UC), Crohn's disease (CD) and indeterminate colitis (IC) or IBD unclassified (IBDU).

Crohn's disease, like many other chronic, inflammatory diseases, can cause a variety of systemic symptoms. Among children, growth failure is common. Many children are first diagnosed with Crohn's disease (pediatric Crohn's disease) based on inability to maintain growth. In addition to systemic and gastrointestinal involvement, Crohn's disease can affect many other organ systems. Inflammation of the interior portion of the eye, known as uveitis, can cause eye pain, especially when exposed to light (photophobia). Inflammation may also involve the white part of the eye (sclera), a condition called episcleritis. Both episcleritis and uveitis can lead to loss of vision if untreated.

Crohn's disease is associated with a type of rheumatologic disease known as seronegative spondyloarthropathy. This group of diseases is characterized by inflammation of one or more joints (arthritis) or muscle insertions (enthesitis). The arthritis can affect larger joints such as the knee or shoulder or may exclusively involve the small joints of the hand and feet. The arthritis may also involve the spine, leading to ankylosing spondylitis if the entire spine is involved or simply sacroiliitis if only the lower spine is involved. The symptoms of arthritis include painful, warm, swollen, stiff joints and loss of joint mobility or function.

Ulcerative colitis is another chronic inflammation of the lining of the gastrointestinal tract. Ulcerative colitis occurs in 35-100 people for every 100,000 in the United States, or less than 0.1% of the population. There is thought to be a bimodal distribution in age of onset, with a second peak in incidence occurring in the 6th decade of life. The disease affects females more than males. The clinical presentation of ulcerative colitis depends on the extent of the disease process. Patients usually present with diarrhea mixed with blood and mucus, of gradual onset. They also may have signs of weight loss, and blood on rectal examination. The disease is usually accompanied with different degrees of abdominal pain, from mild discomfort to severely painful cramps.

Ulcerative colitis is usually confined to the colon (large bowel), with the rectum almost universally being involved. The lining of the affected colon becomes inflamed and is characterized by open sores or ulcers, which bleed and produce pus. Inflammation in the colon also causes the colon to empty frequently, causing diarrhea mixed with blood. Ulcerative colitis is an intermittent disease, with periods of exacerbated symptoms, and periods that are relatively symptom-free. Although the symptoms of ulcerative colitis can sometimes diminish on their own, the disease usually requires treatment to enter a remission.

Ulcerative colitis is associated with a general inflammatory process that affects many parts of the body. Sometimes these associated extra-intestinal symptoms are the initial signs of the disease, such as painful, arthritic knees in a teenager. The presence of the disease cannot be confirmed, however, until the onset of intestinal manifestations.

About half of the people diagnosed with ulcerative colitis have mild symptoms. Others suffer frequent fevers, bloody diarrhea, nausea, and severe abdominal cramps. Ulcerative colitis may also cause problems such as arthritis (seronegative arthritis, ankylosing spondylitis, sacroiliitis), inflammation of the eye (iritis, uveitis, episcleritis), liver disease, and osteoporosis. These complications may be the result of inflammation triggered by the immune system because people with ulcerative colitis have abnormalities of the immune system.

For arthritis, related conditions may include, by way of example, all types of primary inflammatory arthritis, for example, rheumatoid arthritis, psoriatic arthritis, ankylosing spondylitis (previously known as Bechterew's disease or Bechterew syndrome), juvenile idiopathic arthritis (JIA) and gout (metabolic arthritis). In addition to all primary forms of arthritis indicated, the condition diagnosed by the methods of the invention and treated by a biological drug, may include all secondary forms of arthritis, for example, lupus erythematosus, Henoch-Schönlein purpura, haemochromatosis, hepatitis, Wegener's granulomatosis (and many other vasculitis syndromes), Lyme disease and familial mediterranean fever.

In yet some further embodiments, the methods of the invention may be relevant for subjects suffering from arthritis and treated by a biological drug.

It should be appreciated that various forms of arthritis may be generally grouped into two main categories, inflammatory arthritis, and degenerative arthritis, each with different causes. Therefore, according to some specific embodiments, the prognostic methods of the invention may be specifically intended for the diagnosis and/or prognosis of patients suffering from an inflammatory disorder, for example, an inflammatory arthritis, specifically, those treated with at least one biological drug.

Inflammatory arthritis is characterized by synovitis, bone erosions, osteopenia, soft-tissue swelling, and uniform joint space narrowing. More specifically, the hallmarks of joint inflammation are synovitis and erosion of bone. The latter will initially appear as a focal discontinuity of the thin, white, subchondral bone plate. Normally, this subchondral bone plate can be seen even in cases of severe osteopenia, whereas its discontinuity indicates erosion. Although it is true that periarticular osteopenia and focal subchondral osteopenia can appear prior to true bone erosion, it is the presence of bone erosion that indicates definite joint inflammation. As the bone erosion enlarges, osseous destruction extends into the trabeculae within the medullary space. One important feature of inflammatory arthritis relates to the concept of marginal bone erosion. This term is given to bone erosion that is located at the margins of an inflamed synovial joint. This specific location represents that portion of the joint that is intra-articular but not covered by hyaline cartilage. Therefore, early joint inflammation will produce marginal erosions prior to erosions of the subchondral bone plate beneath the articular surface. When looking for bone erosions, multiple views of a joint are essential to profile the various bone surfaces. A second important characteristic of an inflammatory joint process is uniform joint space narrowing. This occurs because destruction of the articular cartilage is uniform throughout the intra-articular space. A third finding of inflammatory joint disease is soft-tissue swelling.

A systemic arthritis is characterized by involvement of multiple joints, and includes two main categories, rheumatoid arthritis and seronegative spondyloarthropathy.

Rheumatoid arthritis (RA), that may be also in some embodiments applicable in the present invention, is a chronic, systemic autoimmune disorder that most commonly causes inflammation and tissue damage in joints (arthritis) and tendon sheaths, together with anemia. It can also produce diffuse inflammation in the lungs, pericardium, pleura, and the sclera of the eye, and also nodular lesions, most common in subcutaneous tissue. It can be a disabling and painful condition, which can lead to substantial loss of functioning and mobility. Serologic markers such as rheumatoid factor and antibodies to cyclic citrullinated peptide are important indicators of rheumatoid arthritis. The radiographic features of rheumatoid arthritis are those of joint inflammation and include particular osteopenia, uniform joint space loss, bone erosions, and soft-tissue swelling. Because of the chronic nature of the inflammation, additional findings such as joint subluxation and subchondral cysts may also be evident.

The seronegative spondyloarthropathy category includes psoriatic arthritis, reactive arthritis, and ankylosing spondylitis, and is characterized by signs of inflammation, multiple joint involvement, and distal involvement in the hands and feet with added features of bone proliferation.

Psoriatic arthritis is a chronic disease characterized by inflammation of the skin (psoriasis) and joints (arthritis).

Males and females are equally likely to suffer from psoriasis. For psoriatic arthritis, males are more likely to have the spondylitic form (in which the spine is affected), and females are more likely to have the rheumatoid form (in which many joints may be involved). Psoriatic arthritis usually develops in people aged 35-55 years. However, it can develop in people of almost any age. Psoriatic arthritis shares many features with several other arthritic conditions, such as ankylosing spondylitis, reactive arthritis, and arthritis associated with Crohn's disease and ulcerative colitis. All of these conditions can cause inflammation in the spine and joints, in the eyes, skin, mouth, and various organs.

Ankylosing spondylitis (AS, previously known as Bechterew's disease, Bechterew syndrome, Marie-Strümpell disease and a form of spondyloarthritis), is usually a chronic and progressive form of arthritis, caused due to inflammation of multiple joints, characteristically the spinal facet joints and the sacroiliac joints at the base of the spine. While ankylosing spondylitis tends to affect these joints and the soft tissues around the spine, other joints may also be affected, as well as tissues surrounding the joints (entheses, where tendons and ligaments attach to bone). Ankylosing spondylitis may also involve areas of the body other than the joints, such as the eyes, heart, and lungs. This disorder frequently results in bony ankylosis (or fusion), hence the term ankylosing, which is derived from the Greek word ankylos, meaning stiffening of a joint. Spondylos means vertebra (or spine) and refers to inflammation of one or more vertebrae.

Ankylosing spondylitis primarily affects young males. Males are four to ten times more likely to have ankylosing spondylitis than females. Most people with the disease develop it at age 15-35 years, with an average age of 26 years at onset.

Reactive arthritis (ReA), another type of seronegative spondyloarthropathy, is an autoimmune condition that develops in response to an infection in another part of the body. Coming into contact with bacteria and developing an infection can trigger reactive arthritis. It has symptoms similar to various other conditions collectively known as “arthritis,” such as rheumatism. It is caused by another infection and is thus “reactive”, i.e., dependent on the other condition. The “trigger” infection has often been cured or is in remission in chronic cases, thus making determination of the initial cause difficult.

The symptoms of reactive arthritis very often include a combination of three seemingly unlinked symptoms, an inflammatory arthritis of large joints, inflammation of the eyes (conjunctivitis and uveitis), and urethritis. It should be indicated that ReA is also known as Reiter's syndrome, it is also known as arthritis urethritica, venereal arthritis and polyarteritis enterica.

It should be appreciated that there are many other forms of inflammatory arthritis, including juvenile idiopathic arthritis, gout and pseudo gout, as well as arthritis associated with colitis or psoriasis. It should be therefore appreciated that the methods of the invention are also applicable for patients suffering of these conditions, specifically, those treated with a biological drug.

More specifically, in some embodiments, the methods of the invention may be applicable for subjects that suffers from juvenile idiopathic arthritis (JIA), treated with a biological drug. JIA, is the most common form of persistent arthritis in children (juvenile in this context refers to an onset before age 16, idiopathic refers to a condition with no defined cause, and arthritis is the inflammation of the synovium of a joint). JIA is a subset of arthritis seen in childhood, which may be transient and self-limited or chronic. It differs significantly from arthritis commonly seen in adults (rheumatoid arthritis), and other types of arthritis that can present in childhood which are chronic conditions (e.g. psoriatic arthritis and ankylosing spondylitis).

It should be appreciated that the methods of the invention may be applicable for subjects suffering from any of the immune-mediated disorders discussed above of any stage or type of the disease or from any of the symptoms detailed above.

It should be further appreciated that the biological drug that may be used in the methods, devices and kits of the invention may be any biological drug used for treating any of the disorders disclosed by the invention.

As indicated above, a subset of immune-mediated diseases applicable in the present invention, is known as autoimmune diseases. As used herein autoimmune diseases arise from an inappropriate immune response of the body against substances and tissues normally present in the body. In other words, the immune system mistakes some part of the body as a pathogen and attacks its own cells. This may be restricted to certain organs (e.g. in autoimmune thyroiditis) or involve a particular tissue in different places (e.g. Goodpasture's disease which may affect the basement membrane in both the lung and the kidney). Autoimmune disease are categorized by Witebsky's postulates and include (i) direct evidence from transfer of pathogenic antibody or pathogenic T cells, (ii) indirect evidence based on reproduction of the autoimmune disease in experimental animals and (iii) circumstantial evidence from clinical clues. To name but a few, autoimmune disease applicable for the methods of the invention include but are not limited to, Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, systemic lupus erythematosus (SLE), multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, juvenile idiopathic arthritis, gout and pseudo gout mixed connective tissue disease, polymyositis, dermatomyositis, vasculitis, polyarteritis nodosa, polymyalgia rheumatica, Wegener's granulomatosis, Behget's syndrome, pemphigus, bullous pemphigoid, dermatitis herpetiformis and fatty liver disease.

In yet some further embodiments, the methods of the invention may be applicable for subjects suffering from an immune-mediated disorder that may be a proliferative disorder, specifically, cancer. As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for non-solid and solid tumors.

Malignancy, as contemplated in the present invention may be selected from the group consisting of carcinomas, melanomas, lymphomas and sarcomas. Malignancies that may find utility in the present invention can comprise but are not limited to hematological malignancies (including leukemia, lymphoma and myeloproliferative disorders), hypoplastic and aplastic anemia (both virally induced and idiopathic), myelodysplastic syndromes, all types of paraneoplastic syndromes (both immune mediated and idiopathic) and solid tumors (including lung, liver, breast, colon, prostate GI tract, pancreas and Karposi). More particularly, the malignant disorder may be hepatocellular carcinoma, colon cancer, melanoma, myeloma, acute or chronic leukemia.

It should be understood that in some further embodiments, when the methods of the invention are used for subjects suffering from cancer, biological drugs used for the treatment of cancer may be applicable herein. A few examples of biological drugs used in the treatment of cancer include, but are not limited to monoclonal antibodies such as Bevacizumab (UNII: 2S9ZZM9Q9V), Cetuximab (UNII: PQX0D8J21J), Panitumumab (UNII: 6A901E312A), Rituximab (UNII: 4F4X42SYQ6), Alemtuzumab (UNII: 3A189DF142V), Ipilimumab (UM: 6T8C155666, Yervoy), that is a check point inhibitor, specifically, a monoclonal antibody that works to activate the immune system by targeting CTLA-4, Trastuzumab (UNII: P188ANX8CK, formerly ticilimumab, CP-675,206) is a fully human monoclonal antibody against CTLA-4, ibritumomab tiuxetan (UNII: 4Q52C550XK), lambrolizumab (formerly MK-3475, Pembrolizumab, Keytruda® UNII: DPT0O3T46P), that is a check point inhibitor, specifically, a humanized antibody that targets programmed cell death (PD-1), Nivolumab (Opdivo® UNII: 31YO63LBSN) is an Fab fragment of an antibody that binds the extracellular domain of PID-1, Atezolizumab (trade name Tecentriq) is a fully humanized, engineered monoclonal antibody of IgG1 isotype against the protein programmed cell death-ligand 1 (PD-L1), Avelumab (trade name Bavencio) is a fully human monoclonal antibody that targets PD-L1, Durvalumab is a human immunoglobulin G1 kappa (IgG1κ) monoclonal antibody that blocks the interaction of PD-L1 with the PD-1 and CD80 (B7.1) molecules, Tremelimumab (formerly ticilimumab; UNII: QEN1X95CIX) that is a check point inhibitor and ado-trastuzumab emtansine (UNII: SE2KH7T06F); therapeutic peptides such as Interferon ct-2b (Intron A® UNII: 43K1W2T1M6) or Interferon P-1b (Betaseron® UNII: TTD90R31WZ); Granulocyte-Macrophage Colony Stimulating Factor such as Sargramostim (Leukine® UNII: 5TAA004E22); IL-2 product such as Aldesleukin (Proleukin® UNII: M89N0Q7EQR).

It should be appreciated that in some embodiments, the methods, devices and kits disclosed by the invention may be applicable for any of the immune-related disorders disclosed by the invention, and may be applicable for determining the amount of nADAs in samples of patients suffering from any of the indicated disorders, and treated with a biological drugs used for any of the immune-related disorders discussed herein. Specifically, any of the drugs indicated by the invention. The invention further provides prognostic methods that may be applicable in determining the treatment regimen of patients suffering from any of the immune-related disorders disclosed by the invention. As used herein, “disease”, “disorder”, “condition” and the like, as they relate to a subject's health, are used interchangeably and have meanings ascribed to each and all of such terms.

It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder, condition or pathology causes a second disease, disorder, condition or pathology.

It should be appreciated that all immune-related disorders as specified herein, may be also applicable in any other aspect of the invention disclosed herein after.

The present invention relates to prognostic methods performed in subjects suffering from immune-mediated disorders, that are treated with at least one biological drug. By “patient”, “individual” or “subject” it is meant any organism who may be affected by the above-mentioned conditions, and to whom the prognostic methods herein described are desired, including humans. More specifically, the methods, devices and kits of the invention described herein after, is intended for mammals. By “mammalian subject” is meant any mammal for which the proposed therapy is desired, including human, equine, canine, and feline subjects, most specifically humans.

As noted above, the subjects are treated with at least one biological drug. The term “treatment” refers to the complete range of therapeutically positive effects of administrating to a subject including inhibition, reduction of, alleviation of, and relief from, a condition known to be treated with a biological drug, for example an immune-mediated disorder as detailed herein. More specifically, treatment or prevention of relapse or recurrence of the disease includes the prevention or postponement of development of the disease, prevention or postponement of development of symptoms and/or a reduction in the severity of such symptoms that will or are expected to develop.

These further include ameliorating existing symptoms, preventing—additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1,2. 5, etc., respectively.

Still further, according to certain embodiments, the method of the invention uses any appropriate biological sample. The term “biological sample” in the present specification and claims is meant to include samples obtained from a mammal subject.

In certain embodiment, the biological sample suitable for the method of the invention may be any one of serum and whole blood sample or any fraction or preparation thereof.

In some embodiments, the sample applicable in the methods, devices and kits of the invention may be a serum sample. In yet some further embodiments, the serum samples used by the invention may be either as naturally obtained from the tested subject or manipulated and prepared. In some embodiments, the serum samples may be concentrated samples. In yet some further embodiments, the serum samples may be diluted and as such, different sera concentrations may be used. In some further embodiments the serum concentration may range between about 0.01% and 100%, More specifically, 0.01%, 0.02%, 0.03%, 0.04%, 0.05%, 0.06%, 0.07%, 0.08%, 0.09%, 0.1%, 0.2%, 0.2%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20%, 25%, 30%, 35%, 40%, 45%, 50%, 55%, 60%, 65%, 70%, 75%, 80%, 85, 90%, 95%, 100% or more. In more specific embodiment, the serum concentration may range between about 1% to about 20%, in yet some further particular embodiments, the serum concentration of the sample may be 5%.

It should be recognized that in certain embodiments a biological sample may be for example, blood cells, blood, serum, plasma, bone marrow, lymph fluid, urine, sputum, saliva, faeces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents. Of particular interest and in some specific embodiment, the sample may be breast milk of nursing mother. In yet some specific embodiments, a biological sample examined by the method of the invention may be a saliva sample. In yet some further specific embodiments, a biological sample may be a urine sample.

As indicated above, step (b) of the methods of the invention involves incubation of the immobilized biological drug with at least one target. In some specific embodiments, that target may be either directly or indirectly labeled with a detectable moiety. In yet some further embodiments, the target may be detected using an affinity molecule, for example, antibody that specifically recognizes and binds the target, when associated with the immobilized drug. It should be appreciated that such antibody or any other affinity molecule applicable herein, may be associated either directly or indirectly with a detectable moiety.

In some further embodiments, the detectable moiety associated with the target used by the method of the invention, or alternatively, associated with an antibody specific to said target, may refer to any chemical moiety that can be used to provide a detectable signal, and that can be attached to a nucleic acid or protein via a covalent bond or noncovalent interaction (e.g., through ionic or hydrogen bonding, or via immobilization, adsorption, or the like). Labels generally provide signals detectable by at least one of fluorescence, chemiluminescence, radioactivity, colorimetry, mass spectrometry, X-ray diffraction or absorption, magnetism, enzymatic activity, electrochemical active compounds, or the like. In some specific embodiments, the detectable moiety may be at least one of conductive, electrochemical, fluorescent, chemiluminescent, enzymatic, radioactive, magnetic, metal, and colorimetric label, or any combinations thereof. Examples of labels useful in connection with the invention, include, but are not limited to at least one of haptens, enzymes, enzyme substrates, coenzymes, enzyme inhibitors, fluorophores, quenchers, chromophores, magnetic particles or beads, redox sensitive moieties (e.g., electrochemically active moieties), luminescent markers, radioisotopes (including radionucleotides), conductive materials, or electrochemical materials that in some embodiments may be suitable for electrochemical detection, specifically, nano- and micro-sized materials, such as gold nanoparticles (GNPs), carbon nanotubes (CNTs), graphene (GR), magnetic particles (MBs), quantum dots (QDs) and conductive polymers, biobarcodes and members of binding pairs. More specific examples include at least one of fluorescein, phycobiliprotein, tetraethyl rhodamine, and beta-galactosidase. Binding pairs may include biotin/Strepavidin, biotin/avidin, biotin/neutravidin, biotin/captavidin, GST/glutathione, maltose binding protein/maltose, calmodulin binding protein/calmodulin, enzyme-enzyme substrate, receptor-ligand binding pairs, and analogs and mutants of the binding pairs. It should be appreciated that the use of tags for labeling the target or any affinity molecule that recognizes the target bound to immobilized drug, is also encompassed by the invention. Thus, in some embodiments, the target may include as a fusion protein a tag that is either recognized by an antibody or by any other affinity molecule. Non-limiting examples for such tag may include His-tag, Flag, HA, myc and the like. Further tags are disclosed herein after in connection with other aspects and embodiments of the invention. It should be further appreciated that the detectable moieties disclosed herein are applicable for any aspect of the invention.

In more specific embodiment, the detectable moiety associated with the target of the method of the invention may be gold or latex label.

As indicated before, in some embodiments thereof, the invention encompasses methods, devices and kits based on electrochemical signal, provided by the label used, and/or by the solid support that further provides conductive materials adapted for transducing and optionally, amplifying or enhancing the electrochemical signal to the electrode. This system therefore may be defined in some embodiments, as an electrochemical biosensor. Thus, in some embodiments, the methods, kits and devices of the invention may be based on electrochemical biosensors. The term “electrochemical biosensor”, as used herein, means an analytical device that consist of a sensitive biological recognition material that is the immobilized drug in the present case, targeting an analyte of interest (the target labeled directly or indirectly with a detectable moiety comprising a conductive material) and a transduction element for converting the recognition process into an amperometric or potentiometric signal.

Still further, electrochemical immunosensors are affinity ligand biosensors based on solid-state devices in which immunochemical reactions occur on a transducer surface to generate an electrochemical signal. The concept of the immunosensor methodology is similar to the conventional ELISA (Enzyme-Linked Immunosorbent Assay), however, in contrast to this immunoassay, modern transducer technology allows the highly sensitive determination of the immuno complex (antibody-antigen, specifically, a biological drug and its target) in different ways. Label-based electrochemical immunosensors require a detectable moiety or marker (label) attached to an antigen (Ag) or antibody (Ab), in the present case, the target to achieve an electron transfer. During the readout, the amount of label is detected and it is assumed to correspond to the concentration of the target analyt.

The detectable moiety may itself be electroactive or able to generate an electroactive product directly on the transducer surface. Moreover, gold nanoparticles (GNPs) are often used to modify the working electrode surface. As labeling a molecule with various agents might influence the efficiency of the binding event, and the yield of the molecule-label coupling reaction is highly variable, the use of label free electrochemical immunosensors has become increasingly popular over the years, and is also encompassed by some embodiments of the invention. Electrochemical impedance spectroscopy (EIS) is the most widely used detection technique that normally requires the addition of an external redox probe. The electron transfer from the detectable moiety to the electrode is affected by the binding event which occurs on the electrode surface.

The different classes of electrochemical biosensors can be divided in two main subclasses: label-based and label free. They are essentially based on the use of screen-printed electrodes (SPEs) coupled with nano- and micro-sized materials, such as gold nanoparticles (GNPs), carbon nanotubes (CNTs), graphene (GR), magnetic particles (MBs), quantum dots (QDs) and conductive polymers, employed to modify the electrode surface and/or as labels to generate highly performing analytical tools.

As indicated above, conductive material used by the methods of the invention in electrochemical-based applications, may be used as detectable moieties and/or as a solid support. In some specific embodiments, GNPs may be used in the methods, kits and devices of the invention as the labeling moiety (detectable moiety) and/or as the solid support.

Thus, in some specific embodiments, GNPs may be used as a solid support, for example in combination with chitosan hydrogel and applied to modify a glassy carbon electrode, forming a composite film (GNPs/Chi). In such embodiments, the biopolymer chitosan may be oxidized (by applying an anodic potential to the electrode) and used as a platform to immobilize the drug of the invention. After incubation of the modified electrode with the sample, the target of the biological drug (e.g., TNF) is added in step (b) of the methods of the invention. Such target may be either directly or indirectly labelled with a detectable moiety, for example, an enzymatic label, such as horseradish peroxidase (HRP). It should be noted that HRP, may be connected in some alternative embodiments to an antibody directed against the target. Upon adding the HRP containing solution, a sandwich electrochemical immuno-sensor is constructed, and the conductivity of GNPs/Chi, facilitates the electron transfer to an electrode, for example, glassy carbon electrode.

In yet some further alternative embodiment, GNPs may be electrodeposited onto the surface of a carbon-based SPE for capturing antibodies i.e. the immobilized biological drug, for enhancing signal. Moreover, to generate a favorable microenvironment for the drug (in terms of activity and stability), an ionic liquid may be employed to modify the electrode surface. Hydrogen peroxide and thionine (reduced form) may be used as HRP substrates and the enzymatic product (thionine oxidized form) may be detected via Cyclic Voltammetry (CV), measuring the reduction peak.

In more specific embodiments, the biological drug suitable for the methods, kits and devices of the invention may be immobilized onto a gold electrode nanostructured with a DNA tetrahedron (DNATH) and the target may be conjugated with ferrocene (FeC-Ab) as a detector. The concentration of the target may be followed by measuring the increase in the square wave voltammetric (SWV) signal corresponding to the oxidation of Fc in the FeC-AbC.

In yet some further embodiments, several biosensors belonging to the label-based electrochemical immuno-sensors using the immuno-magnetic separation with antibody-modified magnetic particles may be employed in the methods, kits and devices of the invention.

In some embodiments, Magnetic particles (MBs) may be used as the solid support of a sandwich immunological complex in which GNPs, conjugated with the target of the biological drug (i.e. TNF) may be used as detectable moiety (labels). At the end of all immunological steps, the modified MBs may be captured on the working electrode of carbon-based SPE, which incorporates a permanent magnet underneath; the electro-reduction of the gold may be measured using Differential pulse voltammetry (DPV).

In other specific embodiments, micro-sized magnetic beads (MMBs) ranging from 1-5 μm or nano-sized magnetic beads (NMBs) ranging from 100-500 nm, may be used for coating the solid support suitable in the methods, kits and devices of the invention.

In some other embodiments, a HRP detectable moiety (label) may be used as electrochemical reporter, instead of GNPs. Thus, in some embodiments, the target of the biological drug may be either directly or indirectly labelled with a detectable moiety, for example, an enzymatic label, such as horseradish peroxidase (HRP).

In some further embodiments, a two-step strategy, which included immuno-magnetic pre-concentration and redox cycling, to amplify the electrochemical signal, may be adopted in the methods, kits and devices of the invention. In particular, MBs modified with the biological drug (that are used as a solid support for the immobilized drug), may be used for separation and pre-concentration of the target. Then, the target conjugated with alkaline phosphatase (ALP) may be employed to form a sandwich complex. Once the binding steps are completed, a mixture of ascorbic acid 2-phosphate (AAP) and tri(2-carboxyethyl) phosphine (TCEP) may be added to the MBs. ALP catalyzed the conversion of AAP to the electroactive ascorbic acid (AA) and, after enzymatic reaction, the solution may be transferred onto a gold SPE and the oxidation of AA may occur. The oxidized AA may then be reduced back by the reductant TCEP, allowing additional signal generation at the electrode surface.

In yet some other embodiments, an ELIME (Enzyme-Linked-Immuno-Magnetic-Electrochemical) assay which involves the formation of a sandwich immunological complex, supported by MBs, and a strip of several magnetized SPEs (localized at the bottom of the wells), connected to a portable instrument and allows multiple simultaneous amperometric measurements, may be employed in the methods, kits and devices of the invention. In some embodiments, the number of magnetized SPEs may be 2, 3, 4, 5, 6, 7, 8, 9, 10, 11, 12, 13, 14, 15, 16, 18 or 20.

In yet some other embodiments, the biological drug may be immobilized on silicon dioxide-coated magnetic Fe3O4 nanoparticles, and the target may be immobilized on gold nanocolloids and detected with a copolymer of an Envision regent (EV, a dextrin amine skeleton anchoring more than 100 molecules of HRP and the target) as a detectable moiety attached to the target. The DPV signal may be monitored after magnetically capturing the MNPs—immuno-complexes on the Surface Plasmon Coupled Emission (SPCE) surface.

In some further embodiments, biobarcodes may be used in the methods, kits and devices of the invention. Nano- and micro-sized particles may be functionalized with unspecific oligonucleotide strands allowing the particles to be “read”. In some embodiments, latex spheres may be modified with ferromagnetic Fe₃O₄ particles. The bio-barcode may be formed by modifying each sphere used as a solid support with the biological drug and single stranded-DNA sequences. The target of the biological drug may be detected by adding the bio-barcodes into well plates containing the target and a biotin-conjugated polyclonal antibody against the target. After formation of a sandwich-type structure, the bio-barcodes may be washed and collected on an avidin-modified SPE, allowing them to be covalently bound to the SPE surface by exploiting the interaction between the electrode-confined avidin and the biotin-tagged polyclonal antibody. The excess of bio-barcodes (without target and then without the biotinylated sandwich complex) may be washed away. Finally, an Ag enhancer solution may be loaded onto the SPE and the amount of bio-barcodes remaining on the electrode surface (proportional to the antigen concentration) may be quantified by Differential Pulse Anodic Stripping Voltammetry (DPASV) measurement of Ag+ in acidic solution.

In some other embodiments, quantum dots (QDs) may be used as label strategy in the methods, kits and devices of the invention.

In some particular embodiments, different quantum dots such as CdS, PbS, CuS may be used. After a dissolution step, the metallic component of the QDs may be released and current peaks may be obtained using Square Wave Anodic Stripping Voltammetry (SWASV), a very effective and widely adopted technique for high sensitivity metal analysis.

In some other embodiments, the biological drug may be attached covalently to a SU-8 substrate, used herein as a solid support a negative epoxy-based photoresist originally developed at IBM Research and ideal to be functionalized with biomolecules without any pretreatment due to the presence of exposed epoxy groups. The target may be labeled with an alkaline phosphatase—conjugated secondary antibody and the oxidation of p-aminophenol generated by hydrolysis of p-aminophenyl phosphate by AP may be measured by differential pulse voltammetry (DPV).

The methods of the invention provide clear strategy to evaluate and measure the neutralizing ADAs in a subject treated with a biological drug. However, in some embodiments thereof, the invention provides in addition means to evaluate the total amount of ADAs (the neutralizing and non-neutralizing ADAs in a subject). Therefore, in some embodiments, the method of the invention may include an additional step for determining the total ADAs in the sample. More specifically, having the drug immobilized to a solid support, the methods of the invention may directly measure the ADAs in the sample that bind the immobilized drug using antibodies labeled by a detectable labeled that specifically recognize and binds the ADAs but not the immobilized drug that in certain embodiments is an antibody. Thus, in case the drug used in the method of the invention is a monoclonal antibody comprising two kappa light chains, the ADAs may be detected by an antibody specifically directed at ADAs that comprise at least one lambda light chain. In such case, the method of the invention further comprises the steps of determining the level of neutralizing and non-neutralizing anti-drug antibodies in the biological sample by providing the incubated sample obtained by step (a) or step (b) with an anti-lambda chain antibody, optionally associated with a second detectable moiety, incubating the labeled anti-lambda chain antibody with the immobilized drug and determining the amount of the second detectable moiety. The amount is indicative of the levels of neutralizing and non-neutralizing lambda chain ADAs present in the biological sample, specifically, the ADAs that comprise at least one lambda light chain. It should be however understood that in case the immobilized drug is a monoclonal antibody that comprises two lambda light chains, this additional step involves the use of an anti-kappa antibody labelled with a detectable label that specifically recognizes and binds ADAs comprising at least one kappa light chain.

As indicated above, in some embodiments thereof, the invention provides in addition to the determination of the nADAs in a sample, also means to evaluate amount of the active biological drug in the same sample, or in another sample of the same subject. In some embodiments, this additional evaluation may be performed using some of the components used in the methods of the invention, e.g., the same labeled target. Thus, in some embodiments, the prognostic method of the invention may further comprise the step of determining the level of an active biological drug in a biological sample of a subject treated with said biological drug. More specifically, the method comprising:

First (a), incubating the sample with at least one non-neutralizing antibody specific for the biological drug. It should be noted that the non-neutralizing antibody is immobilized to a solid support. In some embodiments, the sample used may be the same sample examined by the method of the invention discussed herein above, and as such may be the next step of the method of the invention. Alternatively, any other sample or aliquot of a sample taken from the same subject may be used for this further analysis. The second step (b) involves providing the incubated sample of (a) with a target of said biological drug. It should be noted that the target is associated directly or indirectly with at least one detectable moiety. In some embodiments, the target used herein may be the same target used in the method of the invention, or alternatively, a newly added target.

The next step (c), detecting the detectable moiety to determine the amount of the target. It should be noted that the amount or the target is indicative of the levels of the active drug present in the biological sample and bound to the immobilized non-neutralizing antibody.

Still further, in some further embodiments, the biological drug suitable for the method of the invention may be immobilized indirectly on a solid support via at least one of an anti-drug antibody, anti-Fe fragment antibody and immunoglobulin-binding bacterial proteins Protein A, G, L and any combinations thereof.

Protein A, a 42 kDa protein originally found in the cell wall of the bacteria Staphylococcus aureus; Protein G, expressed in group C and G Streptococcal bacteria much like Protein A; Protein L, isolated from the surface of a bacterium Peptostreptococcus magnus and Protein M, found on the cell surface of a bacterium Mycoplasma genitalium.

In some specific embodiments, the biological drug may be immobilized directly on a solid support. As shown by the examples, the invention provides sensitive methods detecting low amounts of nADAs. In yet some further embodiments, the methods of the invention may allow detection of nADA concentration ranging between about 0.1 to about 1000 ng/ml, specifically, about 0.1, 0.2, 0.3, 0.4, 0.5, 0.6, 0.7, 0.8, 0.9. 1, 5, 10, 15, 20, 25, 30, 35, 40, 45, 50, 55, 60, 65, 70, 75, 80, 85, 90, 95, 100 mg/ml or more, specifically, about 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 ng/ml, or more, specifically, 550, 600, 650, 700, 750, 800, 850, 900, 950, 1000 ng/ml, or more in patient sera. In more specific embodiments, the methods of the invention may allow detection of nADA concentration ranging between about 10 to 500 ng/ml in patient sera. In more specific embodiments, the nADA concentration may be between 100-200 ng/ml in patient sera.

Determining the levels of an active biological drug in a subject is clinically significant since it might enable to predict the clinical outcomes of the treatment with the biological drug. As described below, the invention provides herein prognostic methods based on the determination of the level of an active drug in a subject.

Therefore, in a further aspect, the invention relates to a prognostic method for evaluating and assessing responsiveness of a subject to treatment with a biological drug, for monitoring disease progression and early prognosis of disease relapse. More specifically, such methods may comprise the following steps:

First, in step (a), determining the level of nADA in at least one biological sample of the subject, thereby obtaining an nADA value of the sample.

Next, in step (b), determining if the nADA value obtained in step (a) is any one of positive or negative with respect to a predetermined standard nADA value or to an nADA value in at least one control sample.

Step (c) involves classifying the subject as a non-responder or as a responder. More specifically, a positive nADA value of the sample, may indicate that the subject belongs to a pre-established population associated with non-responsiveness to the biological drug treatment. Specifically, a non-responsive subject. However, a negative nADA value of the sample, may indicate that the subject belongs to a pre-established population associated with responsiveness to the biological drug treatment, specifically, a responsive subject, thereby predicting, assessing and monitoring responsiveness of a mammalian subject to the treatment regimen.

Thus, in some embodiments, the invention provides a method for assessing responsiveness of a subject to a treatment regimen, monitoring disease progression and early prognosis of disease relapse. It should be noted that such method may further comprise the step of calculating the rate of change in the value of neutralizing ADA in the sample in response to the treatment. It should be noted that monitoring a subject may involve determining the levels of the nADAs in at least two or more samples of a subject as will be elaborated herein after.

Thus, in some specific embodiments, the prognostic method of the invention for determining the level of nADA in the at least one biological sample, may be performed by the steps of:

First in step (a), incubating the biological sample with the biological drug immobilized directly or indirectly on a solid support.

In the next step (b), providing the incubated sample of step (a) with a target of the biological drug, and incubating the target with the immobilized drug. As noted above, it should be appreciated that the target may be in some embodiments of the invention, associated with a detectable moiety. In yet some alternative embodiments, an antibody or any other affinity molecule that specifically binds the target that is bound to the immobilized drug, may be used. In some embodiments, such antibody may be directly or indirectly associated with at least one detectable moiety.

Finally, in step (c), determining the amount of the target bound to the immobilized drug. As noted above, this step may be completed either by detecting a detectable moiety associated with the target, or alternatively by detecting a detectable moiety associated with an antibody or any other affinity molecule that recognizes and binds the target when attached to the immobilized drug. The amount may be indicative of the levels of nADAs present in the biological sample. In some embodiments, the amount of the bound target is in reverse correlation with the amount of the nADAs.

It should be understood that determination of a “positive” or alternatively “negative” nADA value with respect to a standard value or a control value may involve in some embodiments comparison of the nADA value of the examined sample as determined or obtained in step (a), with the nADA value obtained or determine for a control sample, or from any established or predetermined nADA value (e.g., a standard value) obtained from a known control (either healthy controls or of subjects suffering from the same immune-related disorder, either responder or non-responder). It should be appreciated that in some embodiments, a sample obtained from the same tested subject, prior to initiation of the treatment with the biological drug, may be used as a control sample. Thus, in some embodiments, “positive” is meant an nADA value that is higher, increased, elevated, over produced in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the nADA value of a healthy or responder control, any other suitable control or any other predetermined standard. Still further, a “negative” nADA value in some embodiments may be a reduced, low, non-existing or lack of nADA in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the nADA value of a non-responder control, any other suitable control or any other predetermined standard. It should be noted that when a sample of the same tested subject before the initiation of the treatment is used as a control, a “negative” result may reflect in some embodiments nADA levels that are either reduced when compared to the levels of the same subject before the initiation of treatment (where production of nADAs is not expected), or within the range of the nADA levels in such control. In most embodiments, no nADA (or almost none) is found before the initiation of the treatment. That is to say that no change has been occurred in the nADA levels upon treatment with the biological drug. Such subject may be therefore classified as a responder. In yet some alternative embodiments, when the tested sample is “positive” when compared to the levels of the same subject before treatment, it means that the levels of nADA (the nADA value) are elevated, increased and enhanced when compared to the control (e.g., the same patient before the initiation of the treatment). in such case, the tested subject may be classified as a non-responder.

Thus, in some embodiments, step (b) of the methods of the invention may involves comparing the nADA value determined and obtained in step (a) with the nADA value of an appropriate control or standard. Wherein the nADA value obtained in the examined sample is “positive”, specifically, higher, enhanced, elevated when compared to a healthy or responder control, the subject is classified as a subject that is non-responsive. It should be noted that in case of existence of nADAs, a “positive” nADA value should be in the range of the nADA value of a control patient classified as a non-responder, or any other cut off value obtained for a population of non-responsive patients. Still further, when the nADA value obtained in the examined sample, is determined as “negative”, specifically, lower, reduced, non-existing nADAs levels when compared to a non-responder control, or any other cut off value obtained for a population of non-responder patients, the subject is classified as a subject that may respond to the biological drug treatment.

In some alternative or optional embodiments, the methods of the invention may further comprise an additional dissociation step. In some embodiments, such dissociation step may be performed prior to step (a). As used herein, the term dissociation step relates to a pretreatment step applied to the biological sample prior to incubation of step (a), performed in conditions suitable for releasing and/or dissociating any complexes that may interfere with the performance or accuracy of the test. In some specific embodiments, such dissociation step may release or dissociate drug/anti-drug antibody complexes, thereby facilitating binding of the nADAs to the immobilized drug. In some particular and non-limiting embodiments the dissociation step may involve pretreating the samples for about 1 to 30 minutes, specifically, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more minutes, more specifically, 15 minutes with at least one dissociating agent. Non limiting examples for an appropriate dissociation agent include any acidic substance, for example, any acid such Acetic acid, Glycine-HCl or any equivalent acid, followed by a neutralizing buffer. In some particular embodiments the acid used as a dissociating agent may be present in an amount of between about 10 mM to about 1000 mM or more. In yet some further specific embodiments, the dissociating agent used may be acetic acid in an amount of between about 300 to 600 mm, specifically, in an amount of 300 mM. Still in some further embodiments, Glycine-HCl may be used as a dissociating agent, specifically, in an amount of 100 mM Glycine-HCl. In some embodiments, following the dissociation step, the dissociating agent may be neutralized by the addition of a neutral butler such as Tris 1M.

It should be understood that any assayed sample may contain more or less biological material than is intended, due to human error and equipment failures. Importantly, the same error or deviation applies to both the biological sample and to any control used. Thus, division of the level of neutralizing ADA value by the control yields a quotient which is essentially free from any technical failures or inaccuracies (except for major errors which destroy the sample for testing purposes) and constitutes a normalized expression value of the nADA level.

Thus, in some embodiments, all the diagnostic methods described by the invention that involve determination of the levels of the nADAs in a sample by measuring the levels of the labeled target or alternatively any other components as discussed above, may further comprise a normalization step. Thus, in certain and specific embodiments, the step of determining the level of detectable biological drug-target in the biological sample to obtain neutralizing ADA level value by the method of the invention may further comprise an additional and optional step of normalization. According to some embodiments, in addition to determination of the level of neutralizing ADA of the invention, the level of detectable biological-target may be determined without incubation with the biological sample, or alternatively, upon incubating with irrelevant drug attached to a solid support.

According to such embodiments, the level of detectable biological-target of the invention obtained in step (c) may be normalized according to a negative control such as the detectable biological-target without incubation with the biological sample or incubation of the sample with a non-relevant drug attached to a solid support, obtained in such additional optional step, thereby obtaining a normalized value.

Optionally, similar normalization may be performed using predetermined standard, when applicable.

Still further, it should be appreciated that in some embodiments an important step in prognostic methods having clinical applicability, such as those defined by the present aspect, after determining the level of nADA (either normalized or not), may be determining whether the value of nADA of the tested sample is within the range of the nADA value of a standard population or of a cutoff value predetermined for such population. This step enables the step of classifying the subject. More specifically, this step involves determining whether the nADA value calculated for the sample, is within the range (e.g., +/−10%) of a cutoff value or a standard value predetermined for a population of responders, or alternatively, within the range of a cutoff value of a population of non-responders.

More specifically, the level of nADA values of the tested samples may be compared to predetermined cutoff values that were predetermined for established populations. As used herein the term “comparing” denotes any examination of the level and/or values obtained in the samples of the invention as detailed throughout in order to discover similarities or differences between at least two different samples.

It should be noted that comparing according to the present invention encompasses the possibility to use a computer based approach.

As described hereinabove, the methods of the invention may refer to a predetermined cutoff value. It should be noted that a “cutoff value”, sometimes referred to simply as “cutoff” herein, is a value that meets the requirements for both high diagnostic sensitivity (true positive rate) and high diagnostic specificity (true negative rate).

In some particular non-limiting embodiments, the cutoff value for true positive measurements i.e. corresponding to patient sera exhibiting nADA may range between about 50 ng/ml to about 100 ng/ml, specifically, about 50, 55, 60, 65, 70, 75, 80, 85, 90, 95, 100 ng/ml, or more. More specifically, in some embodiments the cutoff may range between about 70 ng/ml to about 90 ng/ml. In more specific embodiments, such cutoff value may be 80 ng/ml.

More specifically, the terms “sensitivity” and “specificity” are used herein with respect to the ability of the levels of nADA in a sample as detected by the methods of the present invention, to correctly classify this sample as belonging to a pre-established population associated with responsiveness or alternatively, with non-responsiveness, to treatment with the specific biological drug. In other words, a subject classified as a responsive, or alternatively a non-responsive subject. Simply put, a “positive” nADA value as used herein refers to high nADA value that reflects enhanced nADA, elevated nADA, high nADA levels and even in some embodiments, moderate, but existing expression nADA value. A “negative” nADA value reflects a repressed, low, reduced, or non-existing nADA (lack of nADA). Thus, in some embodiments, when nADAs are produced, a “positive” nADA value of an examined sample may be a value that is higher or within the range of the nADA value of a sample taken from a patient classified as non-responder, or a standard cutoff value calculated for non-responders. A “negative” value would be an nADA value that is lower than the nADA value of the non-responder patients (or standard value, or the value of a control sample). Such value may be within the range of the value of a healthy or responder control sample or a standard value of a healthy or responder population of subject, or subjects that were or treated with the drug.

It should be appreciated that a “control sample” as used herein may reflect a sample of at least one subject (either healthy, a subject that is not affected by the same immune-related disorder, or alternatively, an IBD patient), and preferably, a mixture at least six or more patients.

It should be emphasized that the nature of the invention is such that the accumulation of further patient data may improve the accuracy of the any cutoff values, which may be based on an ROC (Receiver Operating Characteristic) curve generated according to said patient data using analytical software program. The level of neutralizing ADA values are selected along the ROC curve for optimal combination of diagnostic sensitivity and diagnostic specificity which are as close to 100 percent as possible, and the resulting values are used as the cutoff values that distinguish between subjects that respond to treatment, non-responder subjects, subjects in remission or subjects in relapse. The ROC curve may evolve as more and more data values are recorded and taken into consideration, modifying the optimal cutoff values and improving sensitivity and specificity. Thus, it should be appreciated that any initial cutoff values should be viewed as a starting point that may shift as more data allows more accurate cutoff value calculation. In yet some further embodiments, the cutoff value may be dependent on the background found in negative sera as measured with the specific device. In yet some further embodiments, the cutoff value may be dependent on the background found in a specific subject and therefore, may be compared to a previous sample taken from the same subject.

It should be appreciated that “Standard” or a “predetermined standard” as used herein, denotes either a single standard value or a plurality of standards with which the level of the neutralizing ADA from the tested sample is compared. The standards may be provided, for example, in the form of discrete numeric values or is calorimetric in the form of a chart with different colors or shadings for different amounts of bound labeled target; or they may be provided in the form of a comparative curve prepared on the basis of such standards (standard curve).

In certain alternative embodiments, a control sample may be used (instead of, or in addition to, pre-determined cutoff values or standard curves). Accordingly, the values of the nADA detected by the invention in the test sample are compared to the values in the control sample. In certain embodiments, such control sample may be obtained from at least one of a healthy subject, a subject suffering from the same pathologic disorder, a subject that responds to treatment with said medicament and a non-responder subject. It should be noted that in some embodiments a sample of the same tested subject before the initiation of the treatment with the same biological drug, or from another time point of the treatment, may be also used as a control.

Thus, classification of the sample as belonging to a “responsive” or alternatively to a “non-responsive” subjects, or as a sample of a responsive (or responder) or alternatively, a non-responsive (or non-responder) subject, may involve determining whether the value of the nADAs determined by the methods of the invention is within the range of predetermined cutoff value of population of responsive subjects or non-responsive subjects. Still further, in some embodiments, high levels of nADAs may indicate that the tested subject may exhibit non-responsiveness. Thus, in some embodiments, “positive” as defined herein may be determined for subjects having calculated nADAs value (by the methods of the invention), that is within the range of a cutoff value determined for non-responsive population. In the same way, “negative” as used herein, is a subject having an nADA value that is within the range of a cutoff predetermined for a population of responders.

As noted above, the prognostic methods of the invention may be used for predicting responsiveness or non-responsiveness to treatment with the biological drug, in a subject.

The term “response” or “responsiveness” to a certain treatment refers to an improvement in at least one relevant clinical parameter as compared to an untreated subject diagnosed with the same pathology (e.g., the same type, stage, degree and/or classification of the pathology), or as compared to the clinical parameters of the same subject prior to treatment with said biological drug.

The term “non-responder” to treatment with a specific biological drug, refers to a patient not experiencing an improvement in at least one of the clinical parameter and is diagnosed with the same condition as an untreated subject diagnosed with the same pathology (e.g., the same type, stage, degree and/or classification of the pathology), or experiencing the clinical parameters of the same subject prior to treatment with the specific medicament. In yet some further embodiments, non-responder may be a subject experiencing progression and therefor worsening of clinical parameters of the disease.

The term “relapse”, as used herein, relates to the re-occurrence of a condition, disease or disorder that affected a person in the past. Specifically, the term relates to the re-occurrence of a disease being treated with the biological drug, specifically, monoclonal antibodies such as Infliximab as discussed herein. In some embodiments, relapse in case of IBD patients may include manifestation of clinical symptoms, specifically, at least one of diarrhea, vomiting, weight loss, fever, abdominal pain, or any of the clinical symptoms disclosed by the invention.

In case the method of the invention is used for monitoring the disease progression, at least two samples may be obtained from the subjects. These samples may be obtained from different time points, for example, before and after the treatment or between two time points during treatment. Such samples of different time points may be defined herein as “temporally separated samples”. Thus, in certain embodiment, the prognostic method of the invention for monitoring the disease progression may comprise the additional following steps:

In step (d), repeating steps (a) to (c) to obtain an nADA value for at least one more temporally separated sample.

Step (e) involves calculating the rate of change of the nADA value between the temporally-separated samples.

Finally in step (f), determining if the rate of change value obtained in step (e) is positive or negative with respect to a predetermined standard rate of change value or to the rate of change value calculated for nADA in at least one control sample. In other words, determining if there is any change in the nADA value during treatment, when at least two samples taken from at least two time points are compared.

In some embodiments, a positive rate of change value may indicate that the subject belongs to a pre-established non-responsive population associated with at least one of loss of response (LOR), inadequate response, intolerance to the treatment or relapse, thereby monitoring disease progression or providing an early prognosis for disease relapse. In other words, that the subject is a non-responsive subject that displays at least one of LOR, inadequate response, intolerance to the treatment or relapse. More specifically, a “positive” rate of change may reflect, an increase, elevation or enhancement of about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100% or more of the nADA value determined for the sample, when compared between different time points during treatment. Such increase, or in other words, “positive” rate of change, may reflect non-responsiveness, LOR, inadequate response, intolerance to the treatment or relapse of the diseases. It should be noted that in some embodiments, the calculated rate of change may be also compared to the rate of change calculated for healthy or responder control, or alternatively, non-responder controls or any other predetermined standard. In case of positive rate of change, in some embodiments, such rate of change may be either higher or within the range of the rate of change determined for non-responsive control or standard value. In yet some further embodiments, a “negative” rate of change may reflect reduction of about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, or more in the nADA value between treatments (temporally separated samples), and therefore may indicate responsiveness of the subject. Such negative rate of change may be either lower than or within the range of the rate of change of control samples obtained from healthy or responder subjects, or of a standard value for responders.

As indicated above, in accordance with some embodiments of the invention, in order to assess response and determine the rate of change in the level of neutralizing ADA of the invention upon treatment with a specific biological drug, at least two “temporally-separated” test samples must be collected from the treated patient and compared thereafter in order to obtain the rate of change in the level of neutralizing ADA. In practice, to detect a change in the level of neutralizing ADA, at least two “temporally-separated” test samples and preferably more must be collected from the patient.

The level of neutralizing ADA is then determined using the method of the invention, applied for each sample. As detailed above, the rate of change is calculated by determining the ratio between the two values, obtained from the same patient in different time-points or time intervals.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. Non-limiting examples for time intervals relevant in the present invention are disclosed in as Example 2 (see Table 2). For example, this interval may be at least one day, at least two days, at least three days, at least one week, at least two weeks, at least three weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, or more. In yet some further embodiments, time intervals may include a period of at least one month, at least two months, at least three months, at least four months, at least five months, at least one year, two years, three years, four years, five years, six years, seven years, eight years, nine years, ten years, or even more.

More specifically, one sample should be obtained from the examined subject prior to treatment with the specific medicament. Prior as used herein is meant the first time point is at any time before initiation of treatment, ideally several seconds or minutes before initiation of treatment. However, it should be noted that any time point before initiation of the treatment, including hours, days, weeks, months or years, may be useful for this method and is therefore encompassed by the invention. The second time point is collected from the same patient after seconds, minutes, hours, days, weeks, months or even years after initiation of treatment. More specifically, at least 1 second, at least one minute, at least one hour, at least two hours, at least 3 hours, at least 4 hours, at least 6 hours, at least 10 hours, at least 12 hours, at least 24 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 40 days, at least 50 days, at least 60 days, at least 70 days, at least 78 days, at least 80, at least 90 days, at least 100 days, at least 110, at least 120 days, at least 130 days, at least 140 days or at least 150 days or more, after initiation of treatment.

In some embodiments, the second time point may be obtained between 1 hour to 30 month initiation of the treatment. In some other embodiments, the second time point is between 1 week to 54 weeks after initiation of the treatment. In other embodiments, the second time point may be obtained between two weeks to 22 weeks after initiation of the treatment. In yet some other embodiments, the different time points may include 2, 6, 14, 22 and 54 weeks after initiation of the treatment.

Still further, in some embodiments, the first sample may be obtained at the initiation of the treatment (time “0”), just before the application of the biological drug or immediately after the initiation of the treatment, where at least one sample may be obtained after the initiation of the treatment as discussed above. In some embodiments, the sample of time point “0” may be obtained from naïve patient that has been never exposed to any treatment regimen. In other embodiments, the sample of time point “0” may be obtained from a patient that has been treated in the past but has not been treated with the same therapeutic treatment. Still further, the “time point “0” sample may be obtained from a patient that has been treated in the past with the same treatment regimen, for example, 1 year before the current treatment, 6 months before, 5 months before, 4 months before, 3 months before, 2 months before, 1 month before, 3 weeks before, 2 weeks before, or 1 week before the monitored treatment.

In practice, for assessing response to a specific treatment, at least two test samples, for example, in two different time points after the initiation of treatment) must be collected from the treated patient, and preferably more. The level of neutralizing ADA is then determined using the method of the invention, applied for each sample. The rate of change of the levels of neutralizing ADA, is then calculated and determined by dividing the two values obtained from the same patient in different time-points or time intervals one by the other.

It should be noted that it is possible to divide the beginning of the-treatment value by the after treatment value and vice versa. For the sake of clarity, as used herein, the rate of change is referred as the ratio obtained when dividing the value obtained at the later time point of the time interval by the value obtained at the earlier time point (for example before initiation of treatment).

For example, this interval may be at least one day, at least two days, at least three days, at least one week, at least two weeks, at least three weeks, at least one month, at least two months, at least three months, at least four months, at least five months, at least one year, or even more. Permeably the second point is obtained at the earlier time point that can provide valuable information regarding assessing response of the patient to the biological drug treatment.

As appreciated, a predetermined rate of change calculated for a pre-established population as detailed above for example encompasses a range for the rate of change having a low value and a high value, as obtained from a population of individuals including healthy controls, responders and non-responders to said medicament, specifically, the biological drug. Thus a subgroup of responsive patients can be obtained from the entire tested population. In this pre-established responsive population, the low value may be characterized by a low response whereas the high value may be associated with a high response as indicated by regular clinical evaluation. Therefore, in addition to assessing responsiveness to treatment, the rate of change may provide insight into the degree of responsiveness. For example, a calculated rate of change that is closer in its value to the high value may be indicative of a low response and thus although the patient is considered responsive, increasing dosing or frequency of administration may be considered. Alternatively, a calculated rate of change that is closer in its value to the low value may be indicative of a high response, even at times leading to remission and thus the maintenance of the treatment may be considered.

For clarity, when referring to a pre-established population associated with responsiveness, it is meant that a statistically-meaningful group of patients treated with a specific medicament, specifically, the biological drug of the invention was analyzed as disclosed herein, and the correlations between the level of neutralizing ADA values (and optionally other patient clinical parameters) and responsiveness to such treatment was calculated. The population may optionally be further divided into sub-populations according to other patient parameters, for example gender and age.

In yet some other embodiments, the biological drug used by the prognostic method of the invention may be an antibody directed against a biological target.

In certain embodiments, the biological target of the prognostic method of the invention may be a cytokine.

In more specific embodiments, the biological target of the biological drug used by the prognostic method of the invention, may be at least one cytokine. Specifically, TNFα. In such case the drug in some embodiment may be at least one antibody specific for TNFα. In some particular embodiments, the biological drug used by the prognostic method of the invention may be a monoclonal antibody specific for TNFα, specifically, at least one of infliximab, etanercept, adalimumab, certolizumab pegol, golimumab, any biosimilar/s thereof and any combination's comprising the same.

It must be appreciated that any biological drug or any of the biosimilar disclosed by the invention in connection with other aspects, are also applicable in the current aspect as well.

In other embodiments, the subject of the prognostic method of the invention may suffer from an immune-mediated disorder. In some embodiments, an immune-mediated disorder may be at least one of inflammatory disease, an autoimmune disease and a proliferative disorder (specifically, cancer). In some embodiments, the immune-mediated disorder of the prognostic method of the invention may be IBD. In still further some embodiments, the prognostic method of the invention relates to IBD wherein IBD may refer to any one of UC, CD and IC (or IBDU). It must be appreciated that any immune-related disorder disclosed by the invention in connection with other aspects, are also applicable in the current aspect as well.

In some embodiments, the target used by the methods of the invention directly or indirectly associated with at least one detectable moiety. In yet some further embodiments, the detectable moiety may be at least one of fluorescent, chemiluminescent, enzymatic, radioactivity, magnetic, and colorimetric label. More specific embodiments the detectable moiety used by the methods of the invention may be haptens, enzymes, enzyme substrates, coenzymes, enzyme inhibitors, fluorophores, quenchers, chromophores, magnetic particles or beads, redox sensitive moieties (e.g., electrochemically active moieties), luminescent markers, radioisotopes (including radionucleotides), conductive materials, specifically, nano- and micro-sized materials, such as gold nanoparticles (GNPs), carbon nanotubes (CNTs), graphene (GR), magnetic particles (MBs), quantum dots (QDs) and conductive polymers, biobarcodes and members of binding pairs.

In yet some further embodiments, the biological sample suitable for the prognostic method of the invention may be any one of serum and whole blood sample or any fraction or preparation thereof, or any of the samples disclosed herein before in connection with previous aspects of the invention. In certain embodiments, the detectable moiety associated with the target used in step (b) of the prognostic method of the invention may be gold, latex label or alternatively any other detectable moiety as disclosed herein before. It should be appreciated however, that the invention further encompasses the use of antibodies or any other affinity molecules that specifically recognize and bind the target. These antibodies or any other affinity molecules may be directly or indirectly labelled with gold, latex label or any other detectable moiety as disclosed herein before.

As noted above, the methods of the invention provide assessment of the neutralizing ADAs in a subject treated with a biological drug. However, in some embodiments thereof, the invention may further provide means for evaluating the total amount of ADAs (the neutralizing and non-neutralizing ADAs in a subject). Thus, in some embodiments, the method of the invention may include an additional step for determining the total ADAs in the sample. More specifically, having the drug immobilized to a solid support, the methods of the invention may directly measure the ADAs in the sample that bind the immobilized drug using antibodies labeled by a detectable label that specifically recognize and binds the ADAs but not the immobilized drug.

Thus, in some embodiments, where the drug of the prognostic method of the invention is a monoclonal antibody comprising two kappa light chains, the method may further allow detection of any ADA that comprises at least one lambda light chain. In such embodiments, the method may further comprise the steps of determining the level of neutralizing and non-neutralizing anti-drug antibodies in the biological sample by providing the incubated sample of step (a) or step (b) with an anti-lambda chain antibody, optionally associated with a second detectable moiety, incubating the labeled anti-lambda chain antibody with the immobilized drug and determining the amount of the second detectable moiety. The anti-lambda chain antibody will recognize and bind any ADA (having at least one lambda light chain) that is bound to the immobilized drug. The amount of the detectable label is indicative of the levels of neutralizing and non-neutralizing lambda chain ADAs present in the biological sample. It should be however understood that in case the immobilized drug is a monoclonal antibody that comprises two lambda light chains, this additional step involves the use of an anti-kappa antibody labelled with a detectable label that specifically recognizes and binds ADAs comprising at least one kappa light chain. It should be understood that kappa light chain or lambda light chain as referred to herein relate to immunoglobulin light chain.

In yet some other embodiments, the biological drug of the prognostic method of the invention may be immobilized indirectly on a solid support via at least one of an anti-drug antibody, anti-Fe fragment antibody and immunoglobulin-binding bacterial proteins Protein A, G, L and any combinations thereof.

In some other alternative specific embodiments, the biological drug may be immobilized directly on a solid support. It should be understood that any solid support as well as any combination of solid support and detectable moiety disclosed by the invention in connection with other aspects, are also applicable in the current aspect as well.

As providing information on nADAs in the sample, the invention may further provide in some embodiments thereof an alternative or additional version of a prognostic method based on immobilized target, where the bound active biological drug in the sample is measured. Information obtained from both versions may be compared and may even improve clinical significance. Thus, in certain embodiments, the prognostic method of the invention may further comprises the step of determining the level of an active biological drug in a biological sample of a subject treated with the biological drug. In some embodiments, this additional evaluation may be performed using some of the components used in the methods of the invention, e.g., the same labeled target. More specifically, such method may comprise: First (a), incubating the sample with at least one non-neutralizing antibody specific for the biological drug. It should be noted that the non-neutralizing antibody is immobilized to a solid support. In some embodiments, the sample used may be the same sample examined by the method of the invention discussed herein above, and as such may be the next step of the method of the invention. Alternatively, any other sample or aliquot of a sample taken from the same subject may be used for this further analysis. The second step (b) involves providing the incubated sample of (a) with a target of said biological drug. It should be noted that the target is associated directly or indirectly with at least one detectable moiety. In some embodiments, the target used herein may be the same target used in the method of the invention, or alternatively, a newly added target.

The next step (c), detecting the detectable moiety to determine the amount of the target. It should be noted that the amount or the target is indicative of the levels of the active drug present in the biological sample and bound to the immobilized non-neutralizing antibody.

In yet some other embodiments, the biological drug of the prognostic method of the invention may be an antibody directed against a biological target and the biological target may be any molecule disclosed by the invention, in some specific embodiments, the biological target may be at least one cytokine. In yet some further specific embodiments, such target may be at least one of tumor necrosis factor alpha (TNFα).

In certain embodiments, the drug of the prognostic method of the invention may be an antibody specific for a cytokine, specifically TNFα. In more specific embodiments, such drug may be a monoclonal antibody specific for TNFα. Non-limiting examples for such drugs may be at least one of infliximab, etanercept, adalimumab, certolizumab pegol, golimumab, any biosimilar/s and combinations thereof.

In certain embodiment, the biosimilars may be any approved biosimilar of the aforementioned originator biologics.

In yet another aspect, the invention relates to a prognostic method for predicting and assessing responsiveness of a subject to treatment with a biological drug, for monitoring disease progression and early prognosis of disease relapse. Specifically, the method may comprise the following steps: In step (a), determining the level of at least one biological target of at least one biological drug in at least one biological sample of the subject. In some embodiments, the biological sample may be obtained prior to the initiation of the treatment with the biological drug. In this step, the level of the biological target is calculated to obtain a target value of the sample.

In the next step (b), determining if the value of the target obtained in step (a) is any one of positive or negative with respect to a predetermined standard target value or to a target value in at least one control sample.

Step (c) involves classifying the subject as a non-responder or as a responder. More specifically, a positive target value of the sample, indicates that the subject belongs to a pre-established population associated with responsiveness to the biological drug treatment. More specifically, that the subject is a responder or a responsive subject. However, a negative target value of the sample, indicates that the subject belongs to a pre-established population associated with non-responsiveness, specifically, that the subject as a non-responder, or non-responsive subject, to the biological drug treatment, thereby predicting, assessing and monitoring responsiveness of a mammalian subject to the treatment regimen. Thus, in some embodiments, “positive” is meant a target value or the resulting an nADA value calculated, is higher, increased, elevated, overexpressed in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the target value or the nADA value of a healthy or responder control, any other suitable control or any other predetermined standard. It should be noted that the controls are also referred to herein as a pre-established population of responders. Specifically, a population of known responders that were classified as responders using clinical parameters. Still further, a “negative” target or nADA value in some embodiments may be a reduced, low, non-existing or lack of bound target or calculated nADA in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the target or nADA value of a non-responder control, any other suitable control or any other predetermined standard (taken from a pre-established population of known non-responders). It should be noted that when a sample of the same tested subject before the initiation of the treatment is used as a control, a “negative” result may reflect in some embodiments target and therefore nADA levels that are either reduced when compared to the levels of the same subject before the initiation of treatment (where production of nADAs is not expected), or within the range of the nADA levels in such control. In most embodiments, no nADA (or almost none) is found before the initiation of the treatment.

Determining the level of an active biological drug in a subject enables also to guide the medical staff on more accurate and personalized decision regarding the most appropriate regimen for the subject.

Therefore, in a further aspect, the invention relates to a method for determining the treatment regimen of a subject suffering from an immune-mediated disorder. The method may comprise the steps of:

In a first step (a), determining the level of nADA in at least one biological sample of the subject, thereby obtaining an nADA value of the sample;

In step (b), determining if the nADA value obtained in step (a) is any one of positive or negative with respect to a predetermined standard nADA value or to an nADA value in at least one control sample;

In step (c), determining treatment regimen for the subject, wherein:

(i) a positive nADA value of the sample, indicates that the subject belongs to a pre-established population associated with at least one of LOR, inadequate response and intolerance to the biological drug treatment, or in other words that the subject displays or may display at least one of LOR, inadequate response and intolerance to the biological drug treatment, and the subject is recommended not to maintain the treatment or alternatively or additionally recommended to administer at least one immunosuppressive agent; and (ii) a negative nADA value of the sample, indicates that the subject belongs to a pre-established population associated with responsiveness to the biological drug treatment, in other words that the subject is a responder or a responsive subject, and the subject is recommended to maintain the treatment.

In other words, “positive” is meant an nADA value that is higher, increased, elevated, overexpressed in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the nADA value of a healthy or responder control, any other suitable control or any other predetermined standard. Such subject is therefore classified as displaying an LOR, inadequate response and intolerance to the biological drug treatment. In further embodiments such subject is recommended not to maintain the treatment or alternatively or additionally recommended to administer at least one immunosuppressive agent. Still further, a “negative” nADA value in some embodiments may be a reduced, low, non-existing or lack of nADA in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the nADA value of a non-responder control, any other suitable control or any other predetermined standard. Such subject will be classified as a responder to the biological drug treatment, and in some embodiments, the subject is recommended to maintain the treatment.

In some other embodiments, the step of the method of the invention for determining the level of nADA in the at least one biological sample may be performed by the steps of:

Step (a) involves incubating the biological sample with the biological drug immobilized directly or indirectly on a solid support.

In step (b), providing the incubated sample of (a) with a target of the biological drug and incubating the target with the immobilized drug. As noted above, the target may be either associated with a detectable moiety (directly or indirectly), or alternatively, an antibody or any other affinity molecule, may be used.

Step (c), determining the amount of the labeled target bound to the immobilized drug, by detecting the detectable moiety, wherein the amount is indicative of the levels of neutralizing anti-drug antibodies present in the biological sample.

In some embodiments, the methods of the invention may comprise a dissociation step. In yet some further embodiments, such dissociation step may be performed prior to step (a) of incubating the sample with the immobilized drug. In more specific embodiments, the sample may undergo a dissociation step to reduce or eliminate complexes of nADAs and drugs that exist in the patient's sample. In some particular and non-limiting embodiments the dissociation step may involve pretreating the samples for about 1 to 30 minutes, specifically, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more minutes, more specifically, 15 minutes with at least one dissociating agent. Non limiting examples for an appropriate dissociation agent include any acidic substance, for example, any acid such Acetic acid, Glycine-HCl or any equivalent acid, followed by a neutralizing buffer. In some particular embodiments the acid used as a dissociating agent may be present in an amount of between about 10 mM to about 1000 mM or more. In yet some further specific embodiments, the dissociating agent used may be acetic acid in an amount of between about 300 to 600 mm, specifically, the acetic acid used may be in an amount of 300 mM. Still in some further embodiments, Glycine-HCl may be used as a dissociating agent. In certain specific embodiments an amount of 100 mM Glycine-HCl may be used. As indicated above, following the dissociation step, the dissociating agent may be neutralized by the addition of a neutral buffer such as Tris 1M.

In some embodiments, the biological drug used by the methods of the invention may be an antibody directed against a biological target. In yet some further embodiments, the biological target may be a cytokine in more specific embodiments, such cytokine may be TNFα. Thus, in some embodiments, the drug used by the methods of the invention may be at least one of infliximab, etanercept, adalimumab, certolizumab pegol, golimumab, ustekinumab, any biosimilar/s and any combination's thereof.

In yet some further embodiments the target used by the methods of the invention may be directly or indirectly associated with at least one detectable moiety. In more specific embodiments, such detectable moiety may be at least one of conductive, fluorescent, chemiluminescent, enzymatic, radioactive, magnetic, and colorimetric label, or any combinations thereof. It should be noted that any of the detectable moieties disclosed by the invention in connection with other aspects are also applicable in the present methods.

Still further, in some embodiments, the methods of the invention may be particularly applicable for determining the treatment regimen of a subject suffering from an immune-mediated disorder, specifically, an immune-mediated disorder may be at least one of inflammatory disease, an autoimmune disease and a proliferative disorder (specifically, cancer). In yet some further specific embodiments, the immune-mediated disorder may be an inflammatory disorder, specifically, IBD. In some embodiments, the method of the invention may further comprise the step of determining the level of an active biological drug in a biological sample of a subject treated with said biological drug. More specifically, the method may further comprise the following steps. First (a), incubating the sample with at least one non-neutralizing antibody specific for the biological drug. It should be noted that the non-neutralizing antibody is immobilized to a solid support. In some embodiments, the sample used may be the same sample examined by the method of the invention discussed herein above, and as such may be the next step of the method of the invention.

Alternatively, any other sample or aliquot of a sample taken from the same subject may be used for this further analysis. The second step (b) involves providing the incubated sample of (a) with a target of said biological drug. It should be noted that the target is associated directly or indirectly with at least one detectable moiety. In some embodiments, the target used herein may be the same target used in the method of the invention, or alternatively, a newly added target.

The next step (c), detecting the detectable moiety to determine the amount of the target. It should be noted that the amount or the target is indicative of the levels of the active drug present in the biological sample and bound to the immobilized non-neutralizing antibody.

The invention also relates to applications that may be commercialized such as devices or kits enabling detection of levels of nADAs in a biological sample of a subject treated with the biological drug.

Therefore, in yet another aspect, the invention relates to a device for detecting nADAs in a biological sample of a subject treated with the biological drug. More specifically, the device may comprise:

In a first component (a), a labeling composition comprising a biological target of the biological drug, the target specifically recognizes and binds the biological drug. It should be appreciated that in some embodiments, the target provided may be associated either directly or indirectly with a detectable moiety. In yet some further embodiments, a specific antibody that recognizes such target when bound to the immobilized drug, may be further used.

A second component of the device of the invention (b) may be a capture-composition comprising the biological drug immobilized directly or indirectly on a solid support. and a third component (c), comprises a solid support suitable for the reception and transport of the biological sample.

Devices particularly suited for commercial uses in “easy to use” formats for detection and quantification of nADAs in a biological sample may be such as lateral flow system, known also as a “strip test”.

Thus, in more specific embodiment, the device of the invention may be in some embodiments, in the form of a lateral flow device comprising:

a. a solid support suitable for the reception and transport of the biological sample;

b. a labeling composition comprising a biological target of the biological drug. The target specifically recognizes and binds the biological drug. It should be appreciated that in some embodiments, the target provided may be a “labeled target” associated with a detectable moiety. In yet some further embodiments, a specific antibody that recognizes such target when bound to the immobilized drug, may be further used. More specifically, the labeling composition may be located in a predetermined specific initiation zone in the flow path from the sample application zone to the capture zone in the solid support; and

c. a capture-composition comprising the biological drug immobilized directly or indirectly on a solid support, the capture-composition is attached to the solid support in a predetermined location in an termination zone in the solid support.

By “lateral flow” it is meant that the examined sample may be placed on a test strip consisting of a bibulous, chromatographic or other porous material and the sample is wicked laterally through of the test strip by capillary action, coincidentally reacting with various reagents in the strip. The scope of the invention is not limited with respect to the direction of the sample movement through the test strip.

Lateral flow tests are devices intended to detect and/or quantify the presence (or absence) of a target analyte in a sample (matrix). In the present invention the labeled target of the biological drug is quantified, and binding thereof to the immobilized drug that serves as the capture composition, depends on the amount of the nADAs in the tested sample. Specifically, high amount of nADAs in the sample will result in reduced binding of the labeled biological target to the immobilized drug. Many commonly used lateral flow tests are suitable for medical diagnostics either for home testing, point of care testing, or laboratory use. Often produced in a dipstick format, lateral flow tests are a form of immunoassay in which the test sample flows along a solid porous substrate via capillary action. In some cases, after the sample is applied to the test it encounters a colored reagent which mixes with the sample and transits with it in the substrate, encountering lines or zones which have been pretreated with a capturing molecule.

In the instant invention, the colored reagent may be the drug-target that may be either directly or indirectly labeled with a colored or otherwise detectable label. The alternative of using a specific antibody that recognizes said target, is also encompassed by the invention. Depending upon the analytes present in the sample, specifically, the nADAs, the colored reagent can become bound to the immobilized drug at the test line or zone. The test line will show as a colored band or spot in positive samples. In this case, a “positive” sample as defined in this aspect of the invention is a sample that display a low or undetectable amount of nADAs that enable binding of the labeled target to the immobilized drug, and therefore, a detectable signal. Such sample may reflect a responsive subject. Most tests are intended to operate on a purely qualitative basis. However it is possible to measure the intensity of the test line to determine the quantity of analyte in the sample. Handheld diagnostic devices known as lateral flow readers are used by several companies to provide a fully quantitative assay result. By utilizing unique wavelengths of light for illumination in conjunction with either CMOS or CCD detection technology, a signal rich image can be produced of the actual test lines. Using image processing algorithms specifically designed for a particular test type and medium, line intensities can then be correlated with analyte concentrations. One such handheld lateral flaw device platform is made by Detekt Biomedical L.L.C. Alternative non-optical techniques are also able to report quantitative assays results. One such example is a magnetic immunoassay (MIA) which, in the lateral flow test form, also allows for getting a quantified result. One may also obtain semi-quantitative result by comparison of signals emitted by the labeled drug-target to the intensity of signal observed in a standard curve, or with any known amount.

For labeling of said lateral flow assays, in principle, any colored particle can be used, however commonly either latex (blue color) or nanometer sized particles of gold (red color) are used. Fluorescent or magnetic labeled particles can also be used, however, these require the use of an electronic reader to assess the test result.

More specifically, the invention further encompasses the application of electrochemical signal and therefore, in some embodiments thereof, the device of the invention may be a device adapted for electrochemical-based signal. Thus, in some embodiments, the device provided by the invention may be provided in the form of electrochemical lateral flow biosensor (ELFB). The ELFB of some embodiments of the invention may comprise the ELFB strip and electronic detector unit. The strip may be placed inside a plastic housing and connected to the external electronic detector unit (receiver), which reads the amperometric signal from the ELFB strip. The electronic detector unit can be any commercially available potentiostat or galvanostat with electrochemical sensor interface, such as Ivium PocketStat, DropSense micro STAT 400, Metrohm Autolab PGSTAT204 and 910 PSTAT mini, Palm|Sense and EmiStat (by Palm|Sense), SP series and SensorStat (by BioLogic), EZStat and PowerStat (by NuVant Systems) and small hand-held PG581 (by Uniscan Instruments) or more appropriately a proprietary device including electronic adaptor chip to a cell phone or any other suitable mobile device.

In yet some further embodiments, the device of the invention may involve the use of a bio-recognition element, that may be the immobilized drug of the invention (within the capture composition), and a labeling composition that may be directly or indirectly associated with a detectable label that may generate or transmit the electrochemical signal. Detectable labels applicable in the device of the invention may include at least one of conductive, fluorescent, chemiluminescent, enzymatic, radioactive, magnetic, and colorimetric label, or any combinations thereof. In more specific embodiments, nano- and micro-sized materials, such as gold nanoparticles (GNPs), carbon nanotubes (CNTs), graphene (GR), magnetic particles (MBs), quantum dots (QDs) and conductive polymers may be particularly applicable in the device of the invention as a detectable moiety and also to modify the solid support. It should be understood that any detectable moiety disclosed by the invention in connection with other aspects of the invention may be also applicable in the present aspect.

Still further, the device of the invention may in some embodiments involve the use of at least one electrode that may be attached or associated to the solid support. Non limiting example for such electrode may include a screen-printed electrode (SPE). The SPE may comprise more than one working electrode. The dual screen-printed electrode (DSPE) with two elliptic working electrodes, a counter electrode and a reference electrode, developed by DropSense, allow simultaneous detection of two different types of antibodies and quantification of their ratio. Alternatively, one of the working electrodes can be used as a control and another one-as a testing electrode.

In yet some further embodiments, to obtain an amperometric signal, the ELFB device comprises an electrochemically active component (EAC). The role of the EAC in electrochemical system is to transfer electrons to the electrode corresponding to its redox potential. A large variety of EACs is available commercially. In order to choose the proper EAC compound for the biosensor applications, one should take into account the following considerations. Firstly, the working electrode potential is relatively low in most of the biological systems. Secondly, the measurements are performed with small volume samples (that means the EAC must be reactive in low amounts). Thirdly, the EAC must be able to bind to the conjugate particles, such as gold nanoparticles or polymeric particles. The examples of EAC, which are commonly used as electrochemical mediators, are Ferrocene, Thionine and Methylene Blue.

As the EAC transfers electrons to the electrode, for example, a screen-printed electrode (SPE) under its reduction potential, the detection efficiency of the SPE depends on the distance between the EAC and the working electrode. Hence, the measurement of the EAC reduction reaction potential enables the detection and quantification of the analyte complex through the immobilized capture drug or the capture composition. As such, compared to the redox enzyme based assays, that are encompassed by some embodiments of the invention, in which the analyte detection is based on the produced amperometric signal by a linked redox enzyme, other alternative embodiments of the invention are based on measurements of the amperometric signal as a result of bringing the EAC close enough to the working electrode to measure the generated current. The latter is proportional to the amount of the analyte (specifically, the labeled target) in the sample.

Lateral flow Tests can operate as either direct or competitive sandwich assays, as in the present invention.

According to some particular embodiments, the device according to the invention may be especially suited to performing any of the methods according to the invention.

In certain embodiments, the biological drug related to the device of the invention may be an antibody directed against a biological target and more specifically the biological target may be at least one of a cytokine. In more specific embodiments, such target may be a cytokine, specifically, tumor necrosis factor alpha (TNFα).

In another embodiment, the drug of the device of the invention may an antibody specific for a cytokine, specifically, TNFα. In such case the drug may be a monoclonal antibody specific for TNFα. In some particular embodiments, such drug may be an antibody specific for TNFα, said drug is at least one of REMICADE® (infliximab), ENBREL® (etanercept), HUMIRA® (adalimumab), CIMZIA® (certolizumab pegol), SIMPONI® (golimumab), any biosimilar thereof, and any combinations of the same.

In yet some further embodiments, the device of the invention may further comprise a second capture-composition comprising at least one non-neutralizing antibody specific for the biological drug immobilized directly or indirectly on a solid support. It should be noted that such additional capture composition, may be used to capture the biological drug that exists in the sample. The same labeling composition of the device of the invention, specifically, the labelled target, may be used also herein to detect the trapped drug bound to the second capture composition.

Thus, in some embodiments, the device of the invention by using two different capturing compositions and a single labeling composition may allow the detection and determination of both, the nADAs in the sample, as well as the active biological drug in the sample.

In yet another aspect of the invention, the invention relates to a kit, specifically, prognostic kit comprising:

(a) a biological drug immobilized directly or indirectly on a solid support;

(b) a biological target of the biological drug (optionally, associated with a detectable moiety). In some embodiments, the kit of the invention may optionally at least one of: (c) instructions for use; (d) standard curves or control samples; (e) at least one anti-lambda chain antibody, optionally associated with a second detectable moiety and (f) at least one non-neutralizing antibody specific for the biological drug. It should be noted that the non-neutralizing antibody is immobilized directly or indirectly on a solid support.

In some embodiments, the biological drug used for the kit of the invention may comprise an antibody directed against a biological target. In further embodiments, the biological target may be a cytokine. In yet some further specific embodiments the cytokine may be TNFα.

More particular embodiments for such drug may include at least one of infliximab, etanercept, adalimumab, certolizumab pegol, golimumab, any biosimilar and any combinations thereof.

In yet some further embodiments the target of the kit of the invention may be directly or indirectly associated with at least one detectable moiety. Still further, such detectable moiety may be at least one of conductive, fluorescent, chemiluminescent, enzymatic, radioactive, magnetic, metal, and colorimetric label, or any combinations thereof.

In some embodiments, the prognostic kits of the invention may comprise any of the devices of the invention.

In some embodiments thereof, the invention further encompasses any of the kits of the invention as described herein, for use in predicting and assessing responsiveness of a subject to treatment with a biological drug, for monitoring disease progression and early prognosis of disease relapse. It should be noted that in some embodiments, the kits of the invention may further comprise any of the reagents, substances or ingredients suitable for performing any of the methods of the invention for detecting nADAs in a biological sample as described above. It should be further appreciated that any of the reagents, substances or ingredients included in any of the methods and kits of the invention may be provided as reagents embedded, linked, connected, attached, placed or fused to any of the solid support materials described above. These reagents and compounds may be further provided in separated containers.

Still further, the invention provides additional methods enabling to determine the level of an active biological drug. As indicated above, in some embodiments, such methods may be either encompassed as further steps by the methods or devices and kits of the invention, or performed in parallel, and provide further information that relates to the treated patient.

Therefore, in yet another aspect, the invention provides a method for determining the level of an active biological drug in a biological sample of a subject treated with a biological drug. More specifically, the method comprising:

In a first step (a), incubating the sample with at least one non-neutralizing antibody specific for the biological drug. It should be noted that the non-neutralizing antibody is immobilized on a solid support. It should be understood that any solid support as discussed by the invention in connection with other aspects, may be also applicable in this method as well. In the next step (b), providing the incubated sample of (a) with a target of the biological drug, it should be noted that in some embodiments target is associated directly or indirectly to at least one detectable moiety. It should be understood that all detectable moieties discussed by the present disclosure in connection with other aspect, are also applicable in the present aspect.

In the next step (c), detecting the detectable moiety to determine the amount of the target. It should be noted that this amount is indicative of the levels of the active drug present in the biological sample and attached to the immobilized non-neutralizing antibody.

As noted above, a non-neutralizing antibody is any antibody directed against the biological drug, that cannot prevent, reduce, decrease or eliminate its binding to the biological target of the drug and therefore cannot attenuate or affect the activity of the biological drug.

In some specific embodiments, the target used by the method of the invention may be labeled directly or indirectly with at least one detectable moiety that may be at least one of conductive, fluorescent, chemiluminescent, enzymatic, radioactive, magnetic, metal, and colorimetric label, or any combinations thereof.

In certain embodiments, the biological drug related to the method of the invention may be an antibody directed against a biological target wherein the biological target is a cytokine.

In other embodiments, the cytokine of the method of the invention may be TNFα and the drug may be a monoclonal antibody specific for TNFα and more specifically the drug may be at least one of infliximab, etanercept, adalimumab, certolizumab pegol, golimumab, any biosimilar/s thereof and any combinations comprising the same.

In more specific embodiments, such biosimilar may include including but are not restricted to infliximab-dyyb, and SB4 etanercept, SB2 infliximab and SB5 adalimumab.

In some specific embodiment, the method of the invention may be applicable for subjects that suffer from an immune-mediated disorder. It should be appreciated that the methods of the invention may be applicable for subject suffering from any of the immune-mediated disorders disclosed by the invention in connection with other aspect of the invention. In some embodiments, an immune-related disorder may be any one of an inflammatory disease, viral infections, an autoimmune disease, metabolic disorders and a proliferative disorder, specifically, at least one of inflammatory disease, an autoimmune disease and a proliferative disorder.

In yet some other specific embodiment, of particular interest, the immune-mediated disorder which is referred by the methods of the invention may be at least one of inflammatory disease, an autoimmune disease and a proliferative disorder (specifically, cancer). In some specific embodiments the immune-mediated disorder may be an inflammatory disorder such as IBD. In further specific embodiments, IBD may be any one of UC, CD and IC, or IBD unclassified (IBDU).

In certain embodiments, the biological sample related to the method of the invention may be any one of serum and whole blood sample or any fraction or preparation thereof.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

Therefore, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

In carrying out the present invention, unless otherwise indicated, conventional techniques of chemistry, molecular biology, biochemistry, protein chemistry, and recombinant DNA technology, may be employed, all of which are within the skill of the person skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%. The terms “comprises”, “comprising”, “includes”, “including”, “having” and their conjugates mean “including but not limited to”. This term encompasses the terms “consisting of” and “consisting essentially of”. The phrase “consisting essentially of” means that the methods, devices and kits may include additional ingredients and/or steps, but only if the additional ingredients and/or steps do not materially alter the basic and novel characteristics of the claimed method, device or kit. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. Disclosed and described, it is to be understood that this invention is not limited to the particular examples, methods steps, and devices or kits disclosed herein as such methods steps and devices or kits may vary somewhat. It is also to be understood that the terminology used herein is used for the purpose of describing particular embodiments only and not intended to be limiting since the scope of the present invention will be limited only by the appended claims and equivalents thereof. It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Reference is now made to the following examples, which together with the above descriptions illustrate the invention in a non-limiting fashion.

Experimental Procedures

Patient Population

Comparative data regarding infliximab pharmacokinetics was retrieved for IBD patients included in a previously reported prospective study of infliximab pharmacokinetics and immunogenicity, in which infliximab levels were gauged using similar ELISA technique at similar time-points [12]. The study was approved by the medical centers' ethics committees and all patients gave a written informed consent.

TABLE 1
Patient's information (of patients providing
the samples presented in Table 3)

| Number of patients | | 36 | |
|---|---|---|---|
| Age, years - median (IQR) | | 37 | (25-49) |
| Disease duration, years - median (IQR) | | 5 | (1-13) |
| Age at diagnosis - median (IQR) | | 26 | (20-36.5) |
| Male/Female ratio | | 0.8 | |
| Treatment duration at sampling time, | | 4 | (1-14) |
| months - median (IQR) | | | |
| Previous treatment with biologics, n (%) | | 3 | (8.3) |
| Crohns Diseased (CD), n (%) | | 24 | (67) |
| Ulcerative colitis (UC), n (%) | | 12 | (33) |
| CD behavior | Inflammatory n (%) | 11 | (46) |
| | Stricturing n (%) | 3 | (13) |
| | Penetrating n (%) | 11 | (46) |
| CD location | Ileal n (%) | 6 | (25) |
| | Ileo-colonic n (%) | 13 | (54) |
| | Colonic n (%) | 3 | (13) |
| UC location | Left sided colitis n (%) | 7 | (58) |
| | Proctitis n (%) | 2 | (17) |
| | Pancolitis n (%) | 3 | (25) |

| | | |
|---|---|---|
| Infliximab trough serum level at time of | 3.45 | (0.6-12.5) |
| sampling μg/mL (median, IQR) | | |

TABLE 2
Patient's information (of patients providing
the samples presented in Table 4)

| Number of patients | | 8 | |
|---|---|---|---|
| Age, years - median (IQR) | | 36.5 | (30.5-55) |
| Disease duration, years - median (IQR) | | 14.5 | (8.5-20.5) |
| Age at diagnosis - median (IQR) | | 23 | (17-32) |
| Male/Female ratio | | 1.7 | |
| Previous treatment with biologics, n (%) | | 3 | (38) |
| CD, n (%) | | 5 | (63) |
| UC, n (%) | | 3 | (37) |
| CD behavior | Stricturing n (%) | 4 | (80) |
| | Penetrating n (%) | 1 | (20) |
| CD location | Ileal n (%) | 1 | (20) |
| | Ileo-colonic n (%) | 4 | (80) |
| UC location | Left sided colitis n (%) | 2 | (67) |
| | Pancolitis n (%) | 1 | (33) |

| | | |
|---|---|---|
| Infliximab 2 weeks trough serum level μg/mL | 13.45 | (7.2-20) |
| (median, IQR) | | |

Clinical Scores

Clinical status was determined by HBI (Harvey-Bradshaw index) for Crohn's disease (CD) and by SCCAI (Simple Clinical Colitis Activity Index) for ulcerative colitis (UC) patients (Higgins P D, et al. Gut 2005; 54:782-8; Harvey R F, et al. Lancet 1980; 1:514). Clinical remission was defined as HBI<5 for CD patients and SCCAI≤3 for UC patients. Clinical response was defined as drop of ≥3 points of the HBI score and a drop of ≥3 points of the SCCAI score for CD and UC patients respectively. Primary non-response was defined as cessation of vedolizumab therapy by week 14, due to lack of clinical response as defined above (Papamichael K, et al. J Crohns Colitis 2016; 10:1015-23).

Elisa Assay for Specific Detection of Only Neutralizing Anti-Drug Antibodies (ADA) Concentration

A standard ELISA plate was coated with 250 ng/ml Infliximab overnight at 4° C. followed by blocking in 1% BSA in PBS for 1 hour at room temperature (RT). Different concentrations of neutralizing antibody (HCA233, BioRad) or non-neutralizing antibody (HCA234, BioRad) were added to the plate for a 1 hour incubation at RT. After washing, the plate was incubated with 1 μg/ml TNFα in blocking buffer for 1 hour at RT. For detection, an HRP labeled anti-TNFα antibody (ab24473, abcam) was added to the plate for 1 hour at RT followed by TMB substrate. TNF binding after incubation with antibodies was compared to the baseline binding in the absence of antibodies.

Specific Detection of Neutralizing ADA Concentration in the Presence of Sera

A standard ELISA plate was coated with 250 ng/ml Infliximab overnight at 4° C. followed by blocking in 1% BSA in PBS for 1 hour at room temperature (RT). A serial dilution (20 ng/ml to 2.5 ng/ml) of the neutralizing antibody (HCA-233, BioRad) was prepared in either 1% BSA in PBS or in 5% pooled negative sera diluted in 1% BSA solution, and added to the plate for a 1 hour incubation at RT. After washing, the plate was incubated with 1 ug/ml TNFα in 1% BSA for 1 hour at RT. For detection, an HRP labeled anti-TNFα antibody was added to the plate for 1 hour at RT followed by TMB substrate.

Elisa Assay for Specific Detection of Infliximab Sera Level Assay Utilizing TNF for Detection

Anti-Infliximab binding antibody HCA-216 (clone AbD19376_MgG, Bio-Rad Laboratories, Inc.) was used to coat a standard ELISA plate (100 μl of 1 ug ml antibody diluted in carbonate buffer was used per well) overnight at 4° C. After washing, the plates were blocked using 150 μl of 1% BSA in PBS for 60 min. at room temp. 100 μl of either standard concentrations of Infliximab or serum samples, diluted 1:50 in 1% BSA, were incubated in duplicates for 60 min. at room temperature. Plates were then washed and incubated with 100 μl of 1.5 ug/ml TNFα (PeproTech, Inc) for another 60 min, at room temp. Finally, 100 μl of an HRP-labelled anti-TNF antibody (ab24473, abeam, UK) was added at a concentration of 70 ng/ml for 60 min. at room temp. After a final washing step, the plates were reacted with tetramethylbenzidine (TMB) substrate. Serum samples from 32 patients were evaluated for drug levels by the routine assay using anti-Fc for Infliximab detection and by the new assay of the invention. The results were read by an ELISA reader and expressed as μg/ml after normalization versus graded concentrations of 3.125-200 ng/ml Infliximab.

### Example 1

Development of a Method for Determining the Levels of Only Specific Neutralizing Anti-Drug Antibodies

Aiming to develop alternative assays to detect the level of active drug in sera, the inventors previously developed a modified ELISA-based antibody assay [6], based on the ability of the neutralizing antibodies to reduce the availability of exogenously added drug Infliximab (IFX) for binding to immobilized target (TNFα). Patients' sera were spiked with exogenous drug, loaded onto an ELISA plate coated with TNFα and the bound drug was quantified. However, although this ELISA-based assays were shown to be of value with respect to predicting loss of response to anti-TNFα drugs, these assays were found to be sensitive to high drug serum levels since free drug in the patient's serum can also bind the plated TNFα and mask ADA neutralization activity.

Thus, in an attempt to overcome this caveat, an improved assay was developed in which the neutralization capacity of the serum is measured in a direct manner (see FIG. 1). In the new technique, the biologic drug is first immobilized either directly or indirectly onto a solid matrix. Serum is then added, allowing anti-drug antibodies to bind the immobilized drug. It should be noted that the target may be either directly or indirectly labeled. After a washing step during which any unbound drug is removed, a labeled form of the target is added (for example TNFα in the case of detecting ADA to anti-TNFα), binding to the immobilized drug. Thereafter, excess unbound target is washed off and the bound target is measured. In the absence of neutralizing antibodies, the anti-antigen binding sites of the drug are free to bind the labeled target, while in the presence of neutralizing antibodies (in contrast to non-neutralizing antibodies) the target binding sites is blocked, preventing it from binding to the drug and therefore a reduced signal is measured. The method was first tested in an ELISA setup, using commercial neutralizing as well as non-neutralizing antibodies. As shown in FIG. 2, a TNFα-binding curve reflecting the presence of increasing amounts of neutralizing antibodies was demonstrated, while the presence of either free drug or non-neutralizing antibodies did not affect TNFα binding. Therefore, the new proposed assay of the invention, clearly enables to overcome drug sensitivity challenges, is tolerant to serum drug and has the advantage of being rapid and readily usable.

Optimizing Sensitivity of the Assay

In the above-described experiment, the assays were performed with 1% BSA in PBS as the diluent. To ensure that the presence of sera does not interfere with the binding of TNF or with the interaction of the neutralizing antibodies with the plated Infliximab, the assay was performed using pooled negative sera diluted 1:20 in 1% BSA in PBS as the antibody diluent. As depicted in FIG. 3, the addition of sera did affect the signal of the bound TNF, but a standard curve was nonetheless still observed.

The FDA recommends that screening and confirmatory ADA assays achieve a sensitivity of at least 100 nanograms per milliliter (ng/mL). Although traditionally FDA has recommended sensitivity of at least 250-500 ng/mL, recent data suggest that concentrations as low as 100 ng/mL may be associated with clinical events. Furthermore, neutralizing antibodies might have a greater impact on drug activity already at lower concentrations. However, it is appreciated that neutralization assays may not always achieve that level of sensitivity.

The inventors therefore evaluated several variations of the assay's methodology:
lowering the amount of Infliximab bound onto the plate—to confer greater impact to the introduced antibodies, yet the bound TNF signal needs to stay detectable.
increasing the concentration of the sera—avoiding dilution of the antibodies increases the ability to detect them; however possible interference of other serum proteins with the measured signal should be evaluated and avoided.

Lowering the Amount of Infliximab Bound onto the Plate.

Different concentrations of plate-bound-Infliximab were tested, ranging from 100 ng/ml to 500 ng/ml in a volume of 100 ul per well in a 96 well plate. Using the same neutralizing antibodies standards, it was noticed that although coating with 100 ng/ml showed the highest reduction in TNF binding, coating with 250 ng/ml could detect 10 ng/ml of neutralizing antibodies while the overall consistency of the curve was better than the one obtained coating with 100 ng/ml. Subsequent Infliximab coating was at 250 ng/ml. Additional conditions were evaluated to ensure optimal blocking and saturation of TNF binding.

Increasing Sera Concentration.

Different sera dilution ratios were tested to determine the limit of the sera concentration that can be used, without increasing the assay's background and affecting its reproducibility. Sera concentrations between 2% and 10% were tested, spiked with the same concentrations of the commercial neutralizing antibody. As shown in FIG. 4, increasing sera concentration, while not affecting the extent of neutralization to a great extent, did decrease the reproducibility of the results.

Following experiments were thus performed at 1:20 dilution (5%). Based on the observation that concentrations of 5-10 ng/ml of the spiked neutralizing antibodies already show decreased TNF binding, it was assumed that a 1:20 dilution enable detection of neutralizing antibodies at a concentration of 100-200 ng/ml in the patients' sera.

Determining a Cutoff for Neutralizing Activity in Naïve Sera

To assess the neutralizing activity background and determine the cutoff for true positive measurements, serum samples from 15 healthy donors were tested, that were never exposed to the drug. The mean antibody concentration measured in these samples was 29 ng/ml and the standard deviation was 16.3. Hence, if the desired confidence level is 99.7% (mean +/−3 standard deviations, assuming a normal distribution) the cutoff is of about 80 ng/ml.

### Example 2

Determining Levels of Only Specific Neutralizing Anti-Drug Antibodies: Validation Step in Patient' Sera and Prediction of Later Loss of Response to the Drug

The test is performed in serum samples from patients treated with Infliximab alongside the appropriate controls. The test is fine-tuned to perform best at the range of the neutralizing antibodies present in patients' sera. The cohort of patients includes those who were already tested with the other neutralizing test previously designed by the inventors as indicated above, in order to allow a comparison between the different methods, as well as additional samples from currently treated patients Immune detection of total antibody levels is also performed using the lambda chain ELISA for comparison. To test the method's tolerance to serum drug level, a fraction of the sera is tested again in the presence of spiked drug, assessing its impact on the results.

The results are compared to those generated with the previous immune assays using patient sera from early treatment points. Statistical analysis is performed to evaluate the agreement between the methods and the ability of the new assay to predict subsequent loss of response and appearance of high antibody titer in patients.

Still further, the novel method of the invention has been next evaluated using a first cohort of sera that comprise three types of samples as indicated below, all negative for anti-drug antibodies when assayed by the conventional “anti-lambda” method, where the anti-drug antibodies were detected using antibodies directed against the lambda light chain of the antibodies. This method however cannot detect anti-drug antibodies that contain a kappa light chain:

1. Samples from patients with moderate to high levels of Infliximab—serum number marked bold in Table 3

2. Samples from patients who developed antibodies detectable by the anti-lambda method (binding antibodies) in their next visit—serum number underlined in Table 3.

3. Samples from patients with declining levels of Infliximab (Infliximab level of 1 ug/ml and below, marked in italics in the central column) who did not develop detectable antibodies—serum number marked as italics in Table 3.

The samples were sorted based on their neutralizing antibodies measurement values. As can be observed in Table 3, measuring neutralizing antibodies does not seem to always precede the appearance of antibodies detectable by the anti-lambda assay. This result could indicate that there is no association between the development of measurable antibodies and earlier appearance of neutralizing antibodies. However, it could be that there is a mechanistic inhibition which prevents the detection of such antibodies. At this stage, the low amounts of antibodies could be bound to the drug which is still available in those serum samples. In such case, the antibodies, though present at the tested time point, are not free to bind and neutralize the assay drug.

TABLE 3
Measuring anti-Infliximab neutralizing antibodies in patients' sera

| Serum No. | Infliximab Level | Neutralizing antibodies ng/ml |
|---|---|---|
| 3105 | MAX | 0 |
| 4465 | MAX | 0 |
| 2566 | MAX | 0 |
| 408 | 18.2 | 0 |
| 3162 | 16.5 | 0 |
| 3234 | 16.4 | 0 |
| 2659 | 16.2 | 0 |
| 3586 | 11.4 | 0 |
| 1992 | 7.0 | 0 |
| 4386 | 6.4 | 0 |
| 1855 | 6.1 | 0 |
| 4460 | 4.1 | 0 |
| 4186 | 2.8 | 0 |
| 1039 | 2.8 | 0 |
| 4171 | 2.6 | 0 |
| 3661 | 0.3 | 0 |
| 1449 | max | 25 |
| 3753 | 0.9 | 25 |
| 4883 | 0.6 | 32 |
| 1564 | 1.6 | 33 |
| 3555 | 0.4 | 43 |
| 1619 | 0.6 | 49 |
| 1534 | 5.4 | 70 |
| 752 | 4.6 | 77 |
| 1285 | 0.2 | 77 |
| 1427 | 0.0 | 81 |
| 3526 | 1.0 | 93 |
| 1617 | 4.3 | 96 |
| 5118 | 0.0 | 100 |
| 1018 | 11.8 | 109 |
| 678 | 0.4 | 119 |
| 2093 | 0.6 | 123 |
| 3267 | 14.5 | 136 |
| 528 | 0.2 | 152 |
| 1308 | 0.0 | 186 |
| 1323 | 0.0 | 323 |

Table fonts: Bold: High drug level, no Ab development in following visits; underlined: Last time point before the detection of antibodies by the anti-Lambda assay; and italics: Declining drug levels without later antibody detection.

The appearance of neutralizing activity could explain most of the samples in which drug levels started dropping without the later appearance of antibodies measured by the anti-Lambda assay (italics in Table 3). This suggests that these patients develop detrimental antibodies, that might benefit from the addition of immunomodulatory drug, which with the existing available assays, would not be considered for their treatment.

Addition of a Dissociation Step

The addition of a dissociation step, releasing the antibodies from the drug and leaving them available for plate-bound drug neutralization, is evaluated. The samples are treated with 300 mM Acetic acid for 15 minutes in order to dissociate any drug-anti-drug-antibodies complexes prior to assess their neutralizing capability. The neutralizing activity is also examined in the following serum samples from the same patients, in order to determine whether the emerging antibodies are neutralizing antibodies. The inventors analyze whether there are patients who do develop antibodies, measurable by the anti-Lambda assay, but do not loose response. These antibodies could be not-neutralizing, with a minor effect on drug activity and efficiency. Such patients might not benefit from the addition of an immunomodulatory drug to their treatment regimen.

Serial Measurements of Neutralizing Activity in Patients' Sera

It was hypothesized that patient-to-patient heterogeneity could be obscuring the appearance and rise of neutralizing antibodies, as the baseline neutralizing capacity of each patient's sera might be different, and thus the standardization by the negative sera could be less appropriate. Serial samples from patients who lost response were therefore tested. The patients did not develop anti-drug antibodies detectable by the lambda assay. As shown in Table 4, a trend of increasing neutralizing antibodies, compared to the baseline level (prior to treatment) was apparent in the patients who lost response. These results therefore establish the feasibility of using the methods of the invention for predicting non-responsiveness in patients treated with a biological drug.

Here also, the need to use a dissociation step is examined in order to learn whether the neutralizing activity is detectable even earlier, when there is still high levels of Infliximab in their sera.

TABLE 4
Serial measurements of neutralizing activity in patients' sera

| Patients losing | | | Lambda | |
|---|---|---|---|---|
| response Week | | | assay | |
| from treatment | Neutralizing | | measured | |
| initiation | antibodies | Remicade | drugs | |
| 0 | −12.78 | NA | NA | Emergent surgery |
| 2 | 41.36 | 7 | 0 | |
| 6 | 25.81 | 7.9 | 0 | |
| 0 | 61.31 | NA | NA | Stopped IFX |
| 2 | 88.21 | Max | 0 | |
| 6 | 70.61 | 9.2 | 0 | |
| 14 | 159.91 | 3.3 | 0 | |
| 22 | 11.06 | 1.8 | 0 | |
| 35 | 46.07 | 0 | 0 | |
| 0 | 92.77 | NA | NA | Shortened interval |
| 2 | 72.77 | 6.7 | 0 | |
| 13 | 107.77 | 0 | 0 | |
| 19 | 170.97 | 0 | 0.4 | |
| 25 | 156.77 | 0 | 0 | |
| 0 | NA | NA | NA | Dose elevation |
| 2 | 18.86 | 5.8 | 0 | before week 35 |
| 21 | 57.12 | 2.5 | 0 | |
| 35 | 83.12 | 1.1 | 0 | |
| 43 | 310.62 | 0 | 4.6 | |
| 0 | 110.02 | NA | NA | Stopped IFX. No |
| 2 | 172.22 | 7.4 | 0 | clinical response |
| 14 | 208.12 | 0 | 0 | |
| 0 | −14.61 | NA | NA | Side effects - |
| 2 | 8.33 | Max | 0 | allergy |
| 6 | 52.61 | 4.2 | 0 | |
| 14 | 51.41 | 1.1 | 0 | |
| 22 | 191.71 | 0 | 0 | |
| 0 | 92.61 | NA | NA | Stopped IFX. No |
| 2 | 172.51 | 7.7 | 0 | clinical response |
| 6 | 0.3 | 5.5 | 0 | |
| 14 | 18 | 0.7 | 0 | |
| 25 | 57.5 | 0.3 | 0 | |
| 44 | 126.5 | 0.1 | 0 | |
| 0 | −15.3 | NA | NA | |
| 2 | −17.4 | Max | 0 | |
| 14 | −12.7 | 0.55 | 0.8 | |

### Example 3

Setting Up a Parallel Drug Level Assay, Utilizing Labeled Target as the Readout and Testing it in Patients' Sera

In parallel to testing the neutralizing antibody assay in serum, a similar format, with the same labeled target, i.e. labeled TNFα is used in order to quantitate serum drug levels more precisely. Measured levels are evaluated for their agreement with the drug level measurement method utilized, namely using anti-Fc to detect drug binding to a target (TNFα) coated ELISA plate.

In the new drug-level assay of the invention, commercial anti-drug antibodies are first immobilized onto a solid matrix. Serum is then added, allowing the immobilized antibodies to capture the drug. After a washing step, a labeled form of the target is added (for example TNFα in the case of detecting Infliximab), binding to the captured drug. Thereafter, excess unbound target is washed off and the bound target is measured as illustrated in FIG. 5.

In this assay, performed by ELISA as a first step, anti-drug binding antibodies (as opposed to neutralizing antibodies) are used for coating. Serum samples are added to the plate, enabling the binding of circulating drug to the bound antibodies. Known standards of the drug are used to create a standard curve and determine accurate sera drug concentrations. After washing steps, TNF is added and bound by the captured drug and then detected by an HRP-labelled anti-TNF antibody. Different conditions were tested to ensure sensitivity and linearity of the results in the range of concentrations typically measured in patients' sera (FIG. 6A). Drug levels were measured in patients' sera using this new assay as well as with assay using anti-Fc for Infliximab detection. The results were found to highly correspond, with a correlation coefficient of 0.96 (FIG. 6B).

### Example 4

Developing Prototypes for a Rapid Lateral Flow Kits Based on the ELISA Set-Up, for the Detection of Neutralizing Antibodies and Drug Concentration

Commercial kits are used to setting up laboratory rapid lateral flow assays. For this purpose, TNFα are conjugated with gold or latex labels to be used as the readout and to evaluate the labeled molecules' ability to bind to the drugs, first in the ELISA setup and then on in the rapid lateral flow setup. The ability of commercial neutralizing antibodies, or antibodies obtained from sera of patients who lost response, spiked into negative unexposed sera to compete with labeled target for binding the immobilized drug in the lateral flow platform, is examined. The accuracy of the assay for drug level measurement is tested using different concentrations of drug spiked into negative sera. Results are compared to the ELISA setup or to commonly used methods for determining patients' drug levels.

### Example 5

Testing the Rapid Lateral Flow Setup in Patients' Sera and in Whole Blood

The rapid lateral flow assay is first tested with patients' sera known for its neutralizing binding capacity and the results are compared to the ELISA based setup results. The method is examined also directly using whole blood. Models for blood samples from patients, with known serum levels of drug and neutralizing antibodies are concretized by fresh whole blood spiked with the drug and commercial neutralizing antibodies at different concentrations and antibody-drug ratios, are used as models. The results are compared to the same spiking in negative sera. After validation of the kit with commercial antibodies, fresh blood samples from patients who were confirmed to have developed neutralizing antibodies are used (following informed consent).

1. A method for determining the level of neutralizing anti-drug antibodies (nADAs) in biological sample of a subject treated with a biological drug, said method comprising:
a. incubating said biological sample with said biological drug immobilized directly or indirectly on a solid support;
b. providing the incubated sample of (a) with a target of said biological drug and incubating the target with said immobilized drug;
c. determining the amount of said target bound to said immobilized drug, wherein said amount is indicative of the levels of neutralizing anti-drug antibodies present in the biological sample.

2. The method according to claim 1, wherein said biological drug is an antibody directed against a biological target.

3. The method according to claim 1, wherein said biological target is a cytokine, optionally, said cytokine is tumor necrosis factor alpha (TNFα).

4. The method according to claim 1, wherein said target is directly or indirectly associated with at least one detectable moiety.

5. The method according to claim 4, wherein said detectable moiety is at least one of conductive, electrochemical, fluorescent, chemiluminescent, enzymatic, radioactive, magnetic, metal, and colorimetric label, or any combinations thereof.

6. The method according to claim 1, wherein said subject is suffering from an immune-mediated disorder, wherein said immune-mediated disorder is at least one of an inflammatory disease, an autoimmune disease and a proliferative disorder, optionally, said inflammatory disorder is an inflammatory bowel disease (IBD).

7. The method according to claim 1, wherein said drug is a monoclonal antibody comprising two kappa light chains and wherein said method further comprises the steps of determining the level of neutralizing and non-neutralizing anti-drug antibodies in said biological sample by providing said incubated sample obtained by step (a) or step (b), with an anti-lambda chain antibody associated with a second detectable moiety, incubating said labeled anti-lambda chain antibody with the immobilized drug and determining the amount of said second detectable moiety, wherein said amount is indicative of the levels of neutralizing and non-neutralizing lambda chain ADAs present in the biological sample.

8. A prognostic method for assessing responsiveness of a subject to treatment with a biological drug, for monitoring disease progression and early prognosis of disease relapse, said method comprising the steps of:
A. determining the level of nADA in at least one biological sample of said subject, thereby obtaining an nADA value of the sample;
B. determining if the nADA value obtained in step (A) is any one of positive or negative with respect to a predetermined standard nADA value or to an nADA value in at least one control sample;
C. classifying said subject as a non-responder or as a responder wherein a positive nADA value of said sample, indicates that said subject is a non-responder to said biological drug treatment, and wherein a negative nADA value of said sample, indicates that said subject is a responder to said biological drug treatment, thereby predicting, assessing and monitoring responsiveness of a mammalian subject to said treatment regimen, wherein determining the level of nADA in said at least one biological sample, is performed by the method of claim 1, said method comprising the steps of:
a. incubating said biological sample with said biological drug immobilized directly or indirectly on a solid support;
b. providing the incubated sample of (a) with a target of said biological drug and incubating the target with the immobilized drug;
c. determining the amount of said target bound to said immobilized drug, wherein said amount is indicative of the levels of nADAs present in the biological sample.

9. The prognostic method according to claim 8, for monitoring the disease progression, the method comprising:
d. repeating steps (a) to (c) to obtain an nADA value for at least one more temporally-separated sample;
e. calculating the rate of change of said nADA value between said temporally-separated samples;
f. determining if the rate of change value obtained in step (e) is positive or negative with respect to a predetermined standard rate of change value or to the rate of change value calculated for nADA in at least one control sample;
Wherein a positive rate of change value indicates that said subject is a non-responsive subject associated with at least one of loss of response (LOR), inadequate response, intolerance to said treatment or relapse, thereby monitoring disease progression or providing an early prognosis for disease relapse.

10. The prognostic method according to claim 8, wherein said biological drug is an antibody directed against a biological target, optionally, said biological target is a cytokine.

11. The prognostic method according to claim 8, wherein said subject is suffering from an immune-mediated disorder, optionally, said immune-mediated disorder is IBD.

12. The prognostic method according to claim 8, wherein said drug is a monoclonal antibody comprising two kappa light chains and wherein said method further comprises the steps of determining the level of neutralizing and non-neutralizing anti-drug antibodies in said biological sample by providing said incubated sample of (a) or (b), with an anti-lambda chain antibody, associated with a second detectable moiety, incubating said labeled anti-lambda chain antibody with the immobilized drug and determining the amount of said second detectable moiety, wherein said amount is indicative of the levels of neutralizing and non-neutralizing lambda chain ADAs present in the biological sample.

13. The prognostic method according to claim 8, further comprising the step of determining the level of an active biological drug in a biological sample of a subject treated with said biological drug, wherein determining the level of an active drug is performed by a method comprising:
a. incubating said sample with at least one non-neutralizing antibody specific for said biological drug, wherein said non-neutralizing antibody is immobilized to a solid support;
b. providing the incubated sample of (a) with a target of said biological drug, wherein said target is associated directly or indirectly with at least one detectable moiety;
c. detecting said detectable moiety to determine the amount of said target, wherein said amount is indicative of the levels of the active drug present in the biological sample.

14. The method according to claim 1, for determining the treatment regimen of a subject suffering from an immune-mediated disorder, said method comprising the steps of:
a. determining the level of nADA in at least one biological sample of said subject, thereby obtaining an nADA value of the sample;
b. determining if the nADA value obtained in step (a) is any one of positive or negative with respect to a predetermined standard nADA value or to an nADA value in at least one control sample;
c. determining treatment regimen for said subject, wherein:
(i) a positive nADA value of said sample, indicates that said subject is associated with at least one of loss of response (LOR), inadequate response and intolerance to said biological drug treatment, and the subject is recommended not to maintain said treatment and/or administration of immunosuppressive agent; and
(ii) a negative nADA value of said sample, indicates that said subject is associated with responsiveness to said biological drug treatment, and the subject is recommended to maintain said treatment, wherein determining the level of nADA in said at least one biological sample, is performed by the method of claim 1.

15. A device for detecting nADAs in a biological sample of a subject treated with said biological drug, the device comprising:
a. a labeling composition comprising a biological target of said biological drug, said target specifically recognizes and binds said biological drug;
b. a capture-composition comprising said biological drug immobilized directly or indirectly on a solid support; and
c. a solid support suitable for the reception and transport of said biological sample.

16. The device according to claim 15, wherein said device is a lateral flow device comprising:
a. a solid support suitable for the reception and transport of said biological sample;
b. a labeling composition comprising a biological target of said biological drug, said target specifically recognizes and binds said biological drug, said labeling composition is located in a predetermined specific initiation zone in the flow path from the sample application zone to the capture zone in said solid support; and
c. a capture-composition comprising said biological drug immobilized directly or indirectly on a solid support, said capture-composition is attached to said solid support in a predetermined location in an termination zone in said solid support, optionally, said biological drug is an antibody directed against a biological target.

17. The device according to claim 15, wherein said device further comprises a second capture-composition comprising at least one non-neutralizing antibody specific for said biological drug immobilized directly or indirectly on a solid support.

18. A kit comprising:
a. a biological drug immobilized directly or indirectly on a solid support;
b. a biological target of said biological drug; and optionally at least one of:
c. instructions for use;
d. standard curves or control samples;
e. at least one anti-lambda chain antibody, optionally associated with a second detectable moiety;
f. at least one non-neutralizing antibody specific for said biological drug, said non-neutralizing antibody is immobilized directly or indirectly on a solid support, optionally, said biological drug is an antibody directed against a biological target, optionally, said biological target is a cytokine.

19. The kit according to claim 18, for predicting and assessing responsiveness of a subject to treatment with a biological drug, for monitoring disease progression and early prognosis of disease relapse.

20. The method of claim 1, for determining the level of an active biological drug in a biological sample of a subject treated with said biological drug, the method comprising:
a. incubating said sample with at least one non-neutralizing antibody specific for said biological drug, wherein said non-neutralizing antibody is immobilized on a solid support;
b. providing the incubated sample of (a) with a target of said biological drug, wherein said target is associated directly or indirectly to at least one detectable moiety;
c. detecting said detectable moiety to determine the amount of said target, wherein said amount is indicative of the levels of the active drug present in the biological sample and attached to the immobilized non-neutralizing antibody.


SEQUENCE LISTING (2 sequences)

SEQ ID NO: 1 - protein, 233 aa, Homo sapiens
  MISC_FEATURE: TNF-alpha Accession number NP_000585.2
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - DNA, 702 bp, Homo sapiens
  misc_feature: TNF-alpha Accession number NM_000594.3
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga